(19) United States
(12) Patent Application Publication
(10) Pub. No.: US 2018/0228811 A1
(43) Pub. Date: Aug. 16, 2018

(54) GDNF INDUCTION FOR THE TREATMENT OF RETINAL DISORDERS

(71) Applicants: THE SCHEPENS EYE RESEARCH INSTITUTE, INC., Boston, MA (US); GLAXOSMITHKLINE INTELLECTUAL PROPERTY DEVELOPMENT LIMITED, Brentwood, Middlesex (GB)

(72) Inventors: Dwight M. Morrow, King of Prussia, PA (US); Kathryn L. McCabe, King Of Prussia, PA (US); Hong Lin, King Of Prussia, PA (US); Petr Y. Baranov, Somerville, MA (US); Michael J. Young, Essex, MA (US)

(21) Appl. No.: 15/534,961

(22) PCT Filed: Dec. 11, 2015
(86) PCT No.: PCT/US2015/065399

§ 371 Date: Jun. 9, 2017

Related U.S. Application Data
(60) Provisional application No. 62/090,913, filed on Dec. 12, 2014.

(51) Int. Cl. A61K 31/55; A61P 27/02; A61P 27/06; A61K 9/00; A61K 45/06; A61K 47/02; A61K 9/10; A61K 47/26; A61K 47/40; A61K 47/10; A61P 25/28

(52) U.S. Cl. CPC A61K 31/55; A61P 27/02; A61P 27/06; A61K 9/0048; A61K 9/0051; A61K 9/0019; A61P 25/28; A61K 47/02; A61K 9/10; A61K 47/26; A61K 47/40; A61K 47/10; A61K 45/06

(57) ABSTRACT

Methods and compositions for glial cell-derived neurotrophic factor (GDNF) induction in the eye, and for treatment and/or prevention of retinal disorders are described.

### RELATED APPLICATIONS

This application claims the benefit of priority under 35 U.S.C. § 119(e) to U.S. Provisional Application No. 62/090,913, filed Dec. 12, 2014, which is incorporated herein by reference in its entirety.

### FIELD OF THE INVENTION

The present invention relates to novel uses of 7-[4-(4-chloro-benzyloxy)-benzenesulfonyl]-8-methoxy-3-methyl-2,3,4,5-tetrahydro-1H-3-benzazepine or a pharmaceutically acceptable salt thereof. More particularly, the invention relates to the use of such compounds for GDNF induction, treatment of retinal disorders, and increasing survival of retinal neurons.

### REFERENCE TO THE SEQUENCE LISTING

This application incorporates-by-reference nucleotide and/or amino acid sequences which are present in the file named “Sequence_Listing_36770-550001 WO.txt,” which is 17.7 kilobytes in size, and which was created Dec. 11, 2015 in the IBM-PC machine format, having an operating system compatibility with MS-Windows, which is contained in the text file filed Dec. 11, 2015 as part of this application.

### BACKGROUND

Retinal degeneration is the deterioration of the retina caused by the progressive and eventual death of the retinal or retinal pigment epithelium (RPE) cells. There are several reasons for retinal degeneration, including artery or vein occlusion, diabetic retinopathy, retrolental fibroplasia/retinopathy of prematurity, or disease (usually hereditary). These may present in many different ways such as impaired vision, night blindness, retinal detachment, light sensitivity, tunnel vision, and loss of peripheral vision to total loss of vision.

### SUMMARY OF THE INVENTION

The disclosure is based, in part, on novel uses of 7-[4-(4-chloro-benzyloxy)-benzenesulfonyl]-8-methoxy-3-methyl-2,3,4,5-tetrahydro-1H-3-benzazepine (Compound I) to induce glial cell-derived neurotrophic factor (GDNF) in the retina and the use of Compound I to treat retinal disorders. The structure of Compound I is:

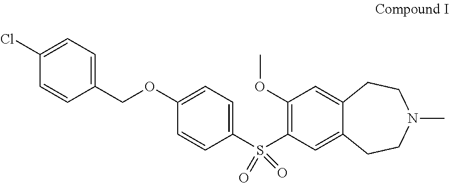 [SMILES: COc1cc2c(cc1S(=O)(=O)c1ccc(OCc3ccc(Cl)cc3)cc1)CCN(C)CC2]

In some aspects, the disclosure provides a method of increasing glial cell-derived neurotrophic factor (GDNF) protein levels in a retina of a subject, the method comprising:

administering Compound I (e.g., a pharmaceutical composition thereof) to a subject (e.g., human subject), wherein the Compound I increases GDNF protein levels in the retina (e.g., as compared to the GDNF protein levels in the retina prior to Compound I administration).

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some aspects, the disclosure provides a method of decreasing retinal neuron loss (e.g., via apoptosis) in a retina of a subject, the method comprising:

administering Compound I (e.g., a pharmaceutical composition thereof) to a subject (e.g., human subject), wherein the Compound I decreases retinal neuron loss in the retina (e.g., as compared to the amount of retinal neuron loss (e.g., number of apoptotic retinal neurons) in the retina prior to Compound I administration or as compared to a control, e.g., the average amount of retinal neuron loss in a cohort of subjects, e.g., a cohort of subjects with the same retinal neurodegenerative disorder).

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, the retinal neuron is a photoreceptor.

In some embodiments, the retinal neuron is a ganglion cell.

In some embodiments, the retinal neuron is a horizontal cell.

In some embodiments, the retinal neuron is an amacrine cell.

In some embodiments, the retinal neuron is a bipolar cell.

In some aspects, the disclosure provides a method of treating a retinal disorder in a subject, the method comprising:

administering Compound I (e.g., a pharmaceutical composition thereof) (e.g., in a therapeutically effective amount) to a subject (e.g., human subject) in need thereof, e.g., thereby treating the retinal disorder.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides a method of preventing a retinal disorder in a subject, the method comprising:

administering Compound I (e.g., a pharmaceutical composition thereof) (e.g., in a therapeutically effective amount) to a subject (e.g., human subject) in need thereof, e.g., thereby preventing the retinal disorder.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides Compound I (e.g., a pharmaceutical composition thereof) (e.g., in a therapeutically effective amount) for use in increasing glial cell-derived neurotrophic factor (GDNF) protein levels in a retina of a subject, as further described herein.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some aspects, the disclosure provides Compound I (e.g., a pharmaceutical composition thereof) (e.g., in a therapeutically effective amount) for use in decreasing retinal neuron loss (e.g., via apoptosis) in a retina of a subject, as further described herein.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, the retinal neuron is a photoreceptor.

In some embodiments, the retinal neuron is a ganglion cell.

In some embodiments, the retinal neuron is a horizontal cell.

In some embodiments, the retinal neuron is an amacrine cell.

In some embodiments, the retinal neuron is a bipolar cell.

In some aspects, the disclosure provides Compound I (e.g., a pharmaceutical composition thereof) (e.g., in a therapeutically effective amount) for use in the treatment of a retinal disorder in a subject, as further described herein.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides Compound I (e.g., a pharmaceutical composition thereof) (e.g., in a therapeutically effective amount) for use in the prevention of a retinal disorder in a subject, as further described herein.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides Compound I for use in the treatment of disorders wherein increasing glial cell-derived neurotrophic factor (GDNF) protein levels in a retina of a subject would be beneficial, as further described herein.

In some aspects, the disclosure provides Compound I for use in the prevention of disorders wherein increasing glial cell-derived neurotrophic factor (GDNF) protein levels in a retina of a subject would be beneficial, as further described herein.

In some aspects, the disclosure provides use of Compound I (e.g., a pharmaceutical composition thereof) (e.g., in a therapeutically effective amount) for the manufacture of a medicament for increasing glial cell-derived neurotrophic factor (GDNF) protein levels in a retina of a subject, as further described herein.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some aspects, the disclosure provides use of Compound I (e.g., a pharmaceutical composition thereof) (e.g., in a therapeutically effective amount) for the manufacture of a medicament for decreasing retinal neuron loss (e.g., via apoptosis) in a retina of a subject, as further described herein.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, the retinal neuron is a photoreceptor.

In some embodiments, the retinal neuron is a ganglion cell.

In some embodiments, the retinal neuron is a horizontal cell.

In some embodiments, the retinal neuron is an amacrine cell.

In some embodiments, the retinal neuron is a bipolar cell.

In some aspects, the disclosure provides use of Compound I (e.g., a pharmaceutical composition thereof) (e.g., in a therapeutically effective amount) for the manufacture of a medicament for the treatment of a retinal disorder in a subject, as further described herein.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides use of Compound I (e.g., a pharmaceutical composition thereof) (e.g., in a therapeutically effective amount) for the manufacture of a medicament for the prevention of a retinal disorder in a subject, as further described herein.

In some embodiments, the retinal disorder comprises a retinal degenerative disorder.

In some embodiments, the retinal degenerative disorder comprises retinitis pigmentosa.

In some embodiments, the retinal degenerative disorder comprises age-related macular degeneration (AMD). In some embodiments, the AMD is dry AMD. In some embodiments, the AMD is wet AMD.

In some embodiments, the retinal degenerative disorder comprises glaucoma.

In some embodiments, the retinal degenerative disorder comprises diabetic retinopathy.

In some embodiments, the retinal degenerative disorder is selected from the group consisting of: retinopathy of prematurity, Usher syndrome, Stargardt's disease, Leber Congenital Amaurosis, choroideremia, Bardet-Biedl syndrome, and Refsum disease.

In some embodiments, the retinal disorder comprises retinal detachment.

In some embodiments, the retinal disorder comprises retinal trauma.

In some embodiments, Compound I (e.g., a pharmaceutical composition thereof) is administered locally.

In some embodiments, Compound I (e.g., a pharmaceutical composition thereof) is administered by ocular delivery.

In some embodiments, Compound I (e.g., a pharmaceutical composition thereof) is administered as a sustained release formulation (e.g., implant or polymeric matrix).

In some embodiments, a second therapy for the retinal disorder is administered in combination with Compound I.

In some aspects, the disclosure provides use of Compound I for the manufacture of a medicament for the treatment of disorders wherein increasing glial cell-derived neurotrophic factor (GDNF) protein levels in a retina of a subject would be beneficial, as further described herein.

In some aspects, the disclosure provides use of Compound I for the manufacture of a medicament for the prevention of disorders wherein increasing glial cell-derived neurotrophic factor (GDNF) protein levels in a retina of a subject would be beneficial, as further described herein.

In some aspects, the disclosure provides a method of increasing the number of retinal neurons in a retina of a subject, the method comprising:

administering Compound I, or a pharmaceutically acceptable salt thereof, to a subject, wherein the Compound I or pharmaceutically acceptable salt thereof increases the number of retinal neurons in the retina.

In some embodiments, Compound I is administered locally.

In some embodiments, Compound I is administered by ocular delivery.

In some embodiments, Compound I is administered as a sustained release formulation.

In some embodiments, the retinal neuron is a photoreceptor. In some embodiments, the retinal neuron is a ganglion cell. In some embodiments, the retinal neuron is a horizontal cell. In some embodiments, the retinal neuron is an amacrine cell. In some embodiments, the retinal neuron is a bipolar cell.

In some embodiments, the number of retinal neurons in the retina increases by at least about 0.5, 0.75, 1, 1.25, 1.5, 1.75, 2, 2.1, or 0.5-2.1 fold compared to the increase in the number of retinal neurons in the retina of a corresponding subject not administered the Compound I or pharmaceutically acceptable salt thereof. In some embodiments, the number of photoreceptors in the retina increases by at least about 0.5, 0.75, 1, 1.25, 1.5, 1.75, 2, 2.1, or 0.5-2.1 fold compared to the increase in the number of photoreceptors in the retina of a corresponding subject not administered the Compound I or pharmaceutically acceptable salt thereof.

In some aspects, the disclosure provides a composition comprising Compound I or a pharmaceutically acceptable salt thereof in an ophthalmically acceptable vehicle.

In some aspects, the disclosure provides an eye drop composition comprising Compound I or a pharmaceutically acceptable salt thereof and a pharmaceutically acceptable carrier within a dispenser suitable for administering a drop of said composition to an eye of a subject.

In some aspects, the disclosure provides a sustained release composition comprising Compound I or a pharmaceutically acceptable salt thereof.

In some embodiments, the composition comprises a polymer.

In some embodiments, the polymer comprises polyethylene glycol (PEG), poly(ethylene vinyl) acetate (EVA), superhydrolyzed PVA, hydroxyalkyl cellulose (HPC), methylcellulose (MC), hydroxypropyl methyl cellulose (HPMC), polycaprolactone, poly(glycolic) acid, poly(lactic) acid, or a polyanhydride.

In some embodiments, the composition comprises polyethylene glycol (PEG) having a molecular weight of at least about 2000, 2500, 3000, 3500, 4000, 5000, 6000, 7000, 8000, or 9000 Daltons (Da), or between about 2000 and about 10000 Da.

In some embodiments, the composition comprises mannitol.

In some embodiments, the composition is a suspension.

In some embodiments, the composition comprises Compound I at a concentration of about 20, 25, 30, 35, 40, 45, 50, 55, 60, 65, 70, 75, or 80 mg/ml.

In some embodiments, the composition comprises about 10, 15, 20, 25, 30, 35, 40, or 10-40 mg/ml PEG.

In some embodiments, the composition comprises water.

In some embodiments, the composition comprises or is in the form of particles. In some embodiments, the size of the particles is:

(a) D₁₀=about 0.5-10.0 μm; D₅₀=about 5-20 μm; D₉₀=about 10-40 μm; or

(b) D₁₀=about 6.8 μm; D₅₀=about 14.2 μm; D₉₀=about 26.5 μm.

In some embodiments, the composition is formulated for injection into a vitreous chamber of a mammal.

In some embodiments, the composition is formulated for injection at a dose of about 250-5000 ug or about 500-1000 ug of the composition into the vitreous chamber of the mammal.

In some embodiments, the mammal is a human. In other embodiments, the mammal is a mammal other than a human.

In some embodiments, the composition is formulated such that an effective amount of Compound I is present in the vitreous chamber at least about 1, 2, 3, 4, 5, 6, 7, 8, 9, 10, 11, 12, 13, 14, or 1-14 days after the composition is injected into the vitreous chamber.

In some aspects, the disclosure provides a composition (e.g., pharmaceutical composition) comprising Compound I, e.g., for ocular administration, e.g., for use as described herein. For example, the composition can be formulated as described herein, e.g., as an aqueous solution or suspension, e.g., comprising the components of the aqueous solution or suspension as detailed herein.

Each embodiment disclosed herein is contemplated as being applicable to each of the other disclosed embodiments. Thus, all combinations of the various elements described herein are within the scope of the invention.

Related apparatus, systems, techniques, and articles are also described.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 (A-E) are plots showing GDNF induction in vitro in mouse eyecups differentiated from miPSc, human retinal pigmented epithelial cells (ARPE19) and human retinal progenitor cells (hRPC) by Compound I solution. Compound I induced GDNF protein in mouse retinal cells, differentiated from iPSc (A), and hRPC (B) and ARPE-19 (C) in dose-dependent manner as measured by ELISA. GDNF is increased in human retinal pigment epithelium cell line, ARPE19 after stimulation with Compound I. Time-course studies in ARPE19 showed that GDNF mRNA (D) induction peaks at 30 minutes after stimulation. GDNF (protein) measured by ELISA (E) shows dose-dependent induction. Data is shown as M±SEM.

FIG. 2 (A-D) are plots showing GDNF induction by Compound I solution in wt and rho−/− mice. GDNF expression is increased in healthy wild-type (wt) retina on both transcript (A) and protein (B) levels 3 hours after intravitreal injection of 10 uM and 100 uM Compound I solution. (C) shows induction of GDNF mRNA in (rho −/−) diseased retina. In the diseased retina (rho −/−) the protein is induced >2 fold by both concentrations (p=0.01 and p=0.11, T test, respectively). GDNF induction is significant on protein (D) for 10 uM Compound I, but not for 100 uM Compound I formulation. Data is shown as M±SEM.

FIG. 3 (A-D) are plots showing pharmacokinetic and efficacy time-course studies in wt mice. (A) and (B) are plots showing Compound I concentration after Compound I suspension injection in wt mice. Compound I concentration was measured by collecting eyes from 1 hour to 14 days. Significant amount of the test compound remained in the eye for at least 2 weeks following single intravitreal injection of Compound I suspension (A). Compound I concentration in mouse eye the vitreoretinal sample (A) decreased significantly over time from 20 mg/g tissue to 5 mg/g tissue after 14 days from a single injection (ANOVA p=0.017). Total Compound I content per eye (ug) (B) dropping from 80 ug to 28 ug indicating that Compound I is still present after 14 days. This amount was sufficient to sustainably upregulate GDNF in the retina at mRNA (p=0.01, ANOVA) (C) and protein (p<0.0001, ANOVA) (D) levels. FIGS. 3 (C) and (D) are plots showing GDNF induction by Compound I suspension in wild-type mice. GDNF expression in the retina is sustainably increased for up to 14 days at mRNA (C) and protein (D) level following intravitreal delivery of Compound I suspension. While mRNA induction remained at 1.2 to 1.8 fold increase vs. vehicle, protein expression increased with time and reached 3.2 fold induction. Data is shown as M±SEM.

FIG. 4 (A-D) are a plot (A) and images (B-D) showing photoreceptor rescue in rho−/− mice by Compound I suspension. Photoreceptor count in 200 micron area shows 1.5 fold increase in cell number following treatment (A). Data is shown as M±SEM. Outer nuclear layer (ONL) is preserved after Compound I treatment (B) compare to vehicle-treated (C) and untreated (D) eyes. The inner nuclear layer (INL), retinal pigment epithelium (RPE) and ganglion cell layer (GCL) remain intact.

FIGS. 5 (A and B) are a plot (A) and images (B) showing photoreceptor rescue in rho−/− mice by Compound I suspension. Single intravitreal injection of Compound I suspension in 4 week old rho−/− mice was sufficient to provide long-term rescue of photoreceptors (10 weeks after treatment). The spider plot of photoreceptor cell number count (A) showed pan-retinal preservation of photoreceptors in the treated group. Data is shown as M±SEM. Observations include significant increase in ONL thickness, and no changes in the retinal pigment epithelium, inner nuclear or ganglion cell layers (B) at 10 weeks after the treatment. Scale bar 100 um.

FIG. 6 is a set of images showing photoreceptor marker expression following Compound I treatment. Slightly increased intensity of recoverin (red) staining is observed in the rho−/− retina 10 weeks after Compound I treatment compared to age-matched controls and vehicle-treated retina. It may be attributed to higher cell number in ONL. No difference was observed in the expression of cone visual pigments (opsin blue and opsin red/green) or length of outer segments (white arrows). DAPI (blue) used as nuclear counterstain. Scale bar 100 um.

FIG. 7 is a set of images showing the expression of glial and bipolar cell markers following Compound I treatment. The immunostaining for GDNF (red) showed the predominant localization of the protein in the outer nuclear layer in all groups: untreated rho−/− (14 weeks of age), vehicle-treated (10 weeks after the injection) and Compound I treated retina (10 weeks after the injection). Glial activation marker Lh×2 and other Muller glia markers—GFAP and GS, as well as bipolar cell marker PKCa were expressed at similar level in all groups. DAPI (blue) used as nuclear counterstain. Scale bar 100 um.

### DETAILED DESCRIPTION

Retinal degenerative disorders, such as age-related macular degeneration and retinitis pigmentosa, cause progressive loss of photoreceptors. In pre-clinical models of retinal dystrophy, treatment with growth and neurotrophic factors including glial cell-derived neurotrophic factor (GDNF), ciliary neurotrophic factor (CNTF), pigment epithelium-derived factor (PEDF), and nerve growth factor (NGF) results in substantial protection and reduction of neuronal loss over time when administered either by direct injection, use of controlled release systems or gene transfer using AAV or plasmids.

The present disclosure is based, in part, on the surprising discovery that 7-[4-(4-chloro-benzyloxy)-benzenesulfonyl]-8-methoxy-3-methyl-2,3,4,5-tetrahydro-1H-3-benzazepine (Compound I) is able to induce GDNF in normal and diseased retina. This induction results in photoreceptor rescue in a mouse model of retinal degeneration.

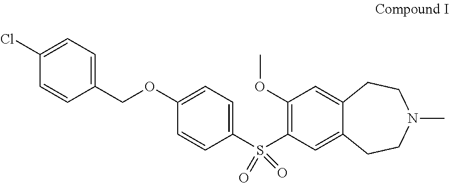 [SMILES: COc1cc2c(cc1S(=O)(=O)c1ccc(OCc3ccc(Cl)cc3)cc1)CCN(C)CC2]

Compound I can be used to increase (e.g., induce) GDNF protein levels in the retina (e.g., as compared to the amount of GDNF protein in the retina prior to Compound I administration). For example, Compound I can increase GDNF protein levels by about 20%, about 30%, about 40%, about 50%, about 60%, about 70%, about 80%, about 90%, about 100%, about 200%, about 300%, about 400%, about 500%, or about 600% as compared to a control, e.g., GDNF protein levels in the retina prior to administration of Compound I.

Compound I can be used to increase (e.g., induce) GDNF mRNA levels in the retina (e.g., as compared to the amount of GDNF mRNA in the retina prior to Compound I administration). For example, Compound I can increase GDNF mRNA levels by about 20%, about 30%, about 40%, about 50%, about 60%, about 70%, about 80%, about 90%, about 100%, about 200%, about 300%, about 400%, about 500%, or about 600% as compared to a control, e.g., GDNF mRNA levels in the retina prior to administration of Compound I.

Compound I may be useful in the treatment of retinal disorders (e.g., that involve the loss (e.g., death, e.g., apoptosis) of retinal neurons), including retinal degenerative disorders, retinal trauma, and retinal detachment.

Compound I may be useful in the treatment of retinal degenerative disorders, such as retinitis pigmentosa, age-related macular degeneration, retinal detachment, glaucoma and other optic neuropathies, and diabetic retinopathy. These disorders can be hereditary or spontaneous.

Compound I may be useful in the treatment of trauma to the retina.

Compound I may be useful in the treatment of retinal detachment.

Compound I may be useful in the prevention of retinal disorders (e.g., that involve the loss (e.g., death, e.g., apoptosis) of retinal neurons), including retinal degenerative disorders, retinal trauma, and retinal detachment.

Compound I may be useful in the prevention of retinal degenerative disorders, such as retinitis pigmentosa, age-related macular degeneration, retinal detachment, glaucoma and other optic neuropathies, diabetic retinopathy, and trauma to the retina, e.g., in a subject who is at risk of developing the retinal degenerative disorder (e.g., carries a mutation that increases risk of developing the retinal degenerative disorder, e.g., a mutation in the rhodopsin gene, which is a cause of retinitis pigmentosa). These disorders can be hereditary or spontaneous.

Compound I may be useful in the prevention of trauma to the retina (e.g., in a subject at risk for trauma to the retina, e.g., who will undergo surgery in or near the retina).

Compound I may be useful in the prevention of retinal detachment (e.g., in a subject at risk for retinal detachment, e.g., who has a retinal tear or other risk factor).

Compound I can be used to decrease retinal neuron loss (e.g., increase retinal neuron survival, and/or e.g., decrease apoptosis of retinal neurons), and/or increase the number of retinal neurons or retinal sensory cells such as photoreceptor cells. For example, Compound I can increase the number of retinal neurons, e.g. photoreceptor cells, that survive (e.g., do not undergo apoptosis) e.g., by about 10%, about 20%, about 30%, about 40%, about 50%, about 60%, about 70%, about 80%, about 90%, about 100%, about 200%, about 300%, or about 400% as compared to the number of retinal neurons that survive in the absence of Compound I administration after some period of time (e.g., 1, 2, 3, 4, 5, 6, 7, 8, 9, 10, 11, or 12 months, or 1, 1.5, 2, 2.5, 3, 3.5, 4, 4.5, 5, 5.5, 6, 7, 8, 9 or 10 years) (e.g., in a given subject), or as compared to a standard, e.g., the average number of retinal neurons in a cohort of subjects (e.g., a population with the same retinal degenerative disorder), e.g., cohort of subjects to whom an agent (e.g., a composition comprising Compound I) was not administered, e.g., after disorder diagnosis or onset of disorder symptoms. The retinal neuron can be a photoreceptor, bipolar cell, ganglion cell, horizontal cell, or amacrine cell. Compound I can increase the survival of one or more of these retinal neuron types. In particular, Compound I can increase the survival and/or number of photoreceptors and ganglion cells.

### 7-[4-(4-Chloro-benzyloxy)-benzenesulfonyl]-8-methoxy-3-methyl-2,3,4,5-tetrahydro-1H-3-benzazepine (Compound I)

7-[4-(4-Chloro-benzyloxy)-benzenesulfonyl]-8-methoxy-3-methyl-2,3,4,5-tetrahydro-1H-3-benzazepine (Molecular Formula: C₂₅H₂₆ClNO₄S) (Compound I) has the structural formula:

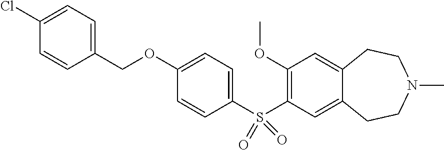 [SMILES: COc1cc2c(cc1S(=O)(=O)c1ccc(OCc3ccc(Cl)cc3)cc1)CCN(C)CC2]

Compound I and its salts are described, e.g., in WO 2003/099786; WO 2005/051916; WO 2006/125622; U.S. Pat. No. 7,504,392, issued Mar. 17, 2009; U.S. Patent Application Publication No. 2007/0275948, published Nov. 29, 2007; and U.S. Patent Application Publication No. 2009/0163475, published Jun. 25, 2009, the entire contents of each of which are hereby incorporated herein by reference. WO 2003/099786 and U.S. Pat. No. 7,504,392 disclose compounds which are 5HT_{2A}, 5HT_{2C}, 5HT₆, D₂ and D₃ agonists, which are useful in the treatment of antipsychotic disorders. One of the compounds disclosed therein is Compound I. WO 2005/051916; WO 2006/125622; U.S. Patent Application Publication No. 2007/0275948, published Nov. 29, 2007; and U.S. Patent Application Publication No. 2009/0163475 disclose maleate and tosylate salts of Compound I, and polymorphic forms thereof.

For example, compounds having the following formula (I) may be used in embodiments of the invention:

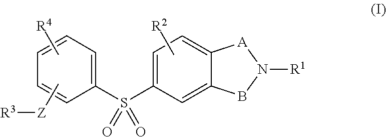

wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁵R⁶, —SO₂NR⁵R⁶, —(CH₂)ₚNR⁵R⁶, —(CH₂)NR⁵COR⁶, optionally substituted aryl ring, optionally substituted heteroaryl ring or optionally substituted heterocyclyl ring;
R³ represents optionally substituted aryl ring or optionally substituted heteroaryl ring;
R⁴ represents hydrogen, hydroxy, C₁₋₆alkyl, C₁₋₆alkoxy, trifluoromethyl, trifluoromethoxy, halogen, —OSO₂CF₃, —(CH₂)ₚC₃₋₆cycloalkyl, —(CH₂)_{q}OC₁₋₆alkyl or —(CH₂)OC₃₋₆cycloalkyl;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
Z represents —CH₂)ᵣX— wherein the —(CH₂)ᵣ— group is attached to R³, or —X(CH₂), wherein
X is attached to R³, and wherein any of the —CH₂— groups may be optionally substituted by one or more C₁₋₆alkyl groups;
X represents oxygen, —NR⁷ or CH₂— wherein the —CH₂ group may be optionally substituted by one or more C₁₋₆alkyl groups;
R⁷ represents hydrogen or C₁₋₆alkyl;
m and n independently represent an integer selected from 1 and 2;
p independently represents an integer selected from 0, 1, 2 and 3;
q independently represents an integer selected from 1, 2 and 3;
r independently represents an integer selected from 0, 1, and 2;

or a pharmaceutically acceptable salt or solvate thereof.

Compounds having the structure of formula (I) and processes for synthesizing such compounds are described in U.S. Pat. No. 7,504,392, issued Mar. 17, 2009, the entire content of which is incorporated herein by reference.

Non-limiting examples of maleate and tosylate salts which are useful in embodiments of the invention include 7-[4-(4-chlorobenzyloxy)benzenesulfonyl]-8-methoxy-3-methyl-2,3,4,5-tetrahydro-1H-3-benzazepinium maleate and a pharmaceutically acceptable solvates thereof and 7-[4-(4-chlorobenzyloxy)benzenesulfonyl]-8-methoxy-3-methyl-2,3,4,5-tetrahydro-1H-3-benzazepinium tosylate and a pharmaceutically acceptable solvates thereof. These compounds and processes for synthesizing such compounds are described in U.S. Patent Application Publication No. 2007/0275948, published Nov. 29, 2007, the entire content of which is incorporated herein by reference. Methods for producing 7-[4-(4-chlorobenzyloxy)benzenesulfonyl]-8-methoxy-3-methyl-2,3,4,5-tetrahydro-1H-3-benzazepinium maleate are also described in U.S. Patent Application Publication No. 2009/0163475, published Jun. 25, 2009, the entire content of which is incorporated herein by reference.

The present invention relates to Compound I or pharmaceutically acceptable salts thereof. Where the following description may refer to Compound I particularly, the invention is intended to include analogous embodiments wherein the compound is 7-[4-(4-chloro-benzyloxy)-benzenesulfonyl]-8-methoxy-3-methyl-2,3,4,5-tetrahydro-1H-3-benzazepine or another pharmaceutically acceptable salt thereof, in particular, hydrochloride, maleate, or tosylate salt thereof. Suitable pharmacologically acceptable salts will be apparent to those skilled in the art and include for example acid addition salts formed with inorganic acids e.g., hydrochloric, hydrobromic, sulfuric, nitric or phosphoric acid; and organic acids e.g., succinic, maleic, malic, mandelic, acetic, fumaric, glutamic, lactic, citric, tartaric, benzoic, benzenesulfonic, p-toluenesulfonic, methanesulfonic or naphthalenesulfonic acid.

In particular embodiments, the compound is the hydrochloride salt of Compound I.

In particular embodiments, the compound is the maleate salt of Compound I.

In other embodiments, the compound is the free base form of Compound I.

Retinal Disorders

Compound I may be useful in the treatment or prevention of a retinal disorder, including retinal degenerative disorders, retinal trauma, and retinal detachment.

In various embodiments, a subject who is afflicted with or suffering from any disorder or disruption of retinal structure or function may be treated using a method or composition of the invention. In certain embodiments, a subject who is “afflicted with” is “suffering from” or is “in need” of treatment for a disorder may be a subject who has been affirmatively diagnosed to have that disorder. In some embodiments, a subject who is in need of preventative or prophylactic treatment for a disorder is a subject who is at risk of developing that disorder.

As used herein, a “symptom” associated with a disorder includes any clinical or laboratory manifestation associated with the disorder, and is not limited to what the subject can feel or observe. For example, symptoms of AMD include a loss of visual acuity, a loss of contrast sensitivity, a perception of colors as less intense or bright, decreased central vision (such as blank or blurry spots in a subject's central vision), visual distortions (such as straight lines appearing wavy or crooked, a doorway or street sign looking lopsided, or objects appearing smaller or farther away than they really are), a well-defined blurry spot or blind spot in a subject's field of vision, and hallucinations of geometric shapes, animals or people; symptoms of retinitis pigmentosa include night blindness and tunnel vision with central vision decreasing as the disease progresses to blindness; symptoms of glaucoma include loss of peripheral or side vision, blurred vision, and the appearance of halos around lights; and symptoms of diabetic retinopathy include spots or dark strings floating in a subject's vision (floaters), blurred vision, fluctuating vision, impaired color vision, dark or empty areas in a subject's vision, and vision loss.

“Treating” (or treatment of) the disorder includes ameliorating at least one symptom of the particular disease or condition, even if the underlying pathophysiology is not affected. The efficacy of the treatment can be evaluated, e.g., as compared to a standard, e.g., improvement in the value or quality of a parameter (e.g., vision, e.g., day vision or night vision) as compared to the value or quality of the parameter prior to treatment. As another example, the efficacy of treatment can be evaluated, e.g., as compared to a standard, e.g., slowing progression of the disorder as compared to a usual time course for the disorder in a cohort that has not been treated or compared to historical data on disorder progression. Treating a disorder also includes slowing its progress; and/or relieving the disorder, e.g., causing regression of the disorder. In some embodiments, the progressive worsening (e.g., the increasing intensity) of a symptom is slowed, reduced, or halted. For example, the loss of retinal neuronal cells, e.g., photoreceptor cells, ganglion, horizontal, amacrine, or bipolar cells, is reduced or halted. In some cases, the number of such cells is increased following therapy/treatment thereby conferring a clinical benefit to the subject.

“Preventing” (or prevention of) a disorder includes stopping a disorder from occurring in a subject, who may be predisposed to the disorder but has not yet been diagnosed as having it. Preventing a disorder also includes delaying the onset of the disorder. The efficacy of the prevention can be evaluated, e.g., as compared to a standard, e.g., delaying onset of the disorder as compared to a usual time of onset for the disorder in a cohort that has not been treated or compared to historical data on disorder onset.

As used herein and depending on the context in which it is used, “therapeutically effective amount” refers to an amount which is effective in reducing, eliminating, treating, preventing or controlling a symptom of a disorder or condition. The term “controlling” is intended to refer to all processes wherein there may be a slowing, interrupting, arresting, or stopping of the progression of the disorders and conditions described herein, but does not necessarily indicate a total elimination of all disease and condition symptoms, and is intended to include prophylactic treatment.

The retinal disorders that may be treated or prevented with the compositions and methods described herein can include the loss (e.g., death, e.g., apoptosis) of one or more types of retinal neurons. Compound I may be used, e.g., to slow or prevent the loss of retinal neurons, thereby treating or preventing the retinal disorder. Compound I may also be used to increase the number of one or more retinal neurons, thereby treating or preventing a retinal disorder.

Aspects of the present invention relate to “neuroprotection.” Depending on context, neuroprotection includes mechanisms and strategies used to protect against neuronal injury. Neuronal injury includes but is not limited to the loss of neurons (such as photoreceptors, ganglion cells, horizontal cells, amacrine cells, or bipolar cells) as a result of a retinal disorder. As used herein, “indirect neuroprotection” includes neuroprotection without the direct administration of protein growth factor or cell that produces a protein growth factor. The administration of Compound I is an example of indirect neuroprotection.

The present invention relates in part to the surprising discovery that Compound I is effective at treating retinal disorders. Without wishing to be bound by any scientific theory, Compound I is a potent neuroprotectant in retinal tissues, with its effects at least partially mediated by GDNF. Compound I increases retinal GDNF protein levels and is sufficient to prevent or treat retinal disorders. Additionally, Compound I protects against the loss of photoreceptors at lower concentrations than other small molecule compounds such as amitriptyline, rasagiline, and valproic. Without wishing to be bound by any scientific theory, Compound I significantly increases GNDF protein levels in retinal tissues after administration. This increase in GNDF rescues or promotes the survival of photoreceptors to a level that was previously achieved with the transplantation GDNF-overexpressing Schwann cells (Keegan et al., Invest. Ophthalmol. Vis. Sci. 44, 3526-3532 (2003)). However, Compound I is a small molecule that can be administered using minimally invasive (such as intravitreal injection) and non-invasive (such as eye drops) techniques. In one non-limiting embodiment shown in Example 2, a single intravitreal injection of a sustained release Compound I formulation is sufficient to rescue photoreceptors in rhodopsin-knockout retinal degeneration mice. These results show that Compound I is effective at treating inherited disorders with a high level of genetic penetrance (in addition to other disorders).

The ability of Compound I to induce GNDF expression in both normal and diseased tissues supports the discovery that it is useful not only for treating subjects afflicted with a disorder, but also in subjects at risk of developing a disorder. Even minimally invasive administration techniques may not be tolerated for subjects with no symptoms of a disorder. The option to administer Compound I non-invasively makes it a valuable and attractive tool for reducing, delaying, or preventing the onset of a disorder in a subject who is not suffering from symptoms of the disorder.

Retinal Degenerative Disorders

Compound I may be useful in the treatment or prevention of a retinal degenerative disorder.

Two of the most commonly-occurring retinal degenerative disorders are retinitis pigmentosa (RP) and age-related macular degeneration (AMD). Retinitis pigmentosa is a heterogenous group of inherited eye disorders that results in degeneration of the photoreceptor cells of the retina, also known as rods and cones, leading to blindness. The origin of RP can be caused by a number of mutations that cause the proteins to either not function, limit the function of the photoreceptor, or to be toxic to the photoreceptors. All three major causes result in photoreceptor death and vision loss. Symptoms include night blindness, tunnel vision with central vision decreasing as the disease progresses to blindness. Over 100 mutations have been implicated in the disease. See, e.g., Hartong et al., Lancet 368:1795-1809 (2006). Age-related macular degeneration (AMD) has two forms: dry and wet. The macula is the name given to the central portion of the retina and is responsible for central, as opposed to peripheral, vision. Both forms of the AMD can lead to a loss of sharpness, brightness, or blank spots in central vision that limit the ability to function in everyday tasks such as reading, writing, and seeing faces. The more common form, dry AMD, is caused by the buildup of cellular debris (drusen) between the retina and the choroid (the layer of the eye beneath the retina), leading to atrophy of photoreceptor cells. The other form of AMD, wet AMD, results from abnormal growth of blood vessels in the choroid. These vessels may leak, resulting in damage to the choroid and the retina, and death of retinal neurons. Other terms for AMD include choroidal neovascularization, subretinal neovascularization, exudative form and disciform degeneration.

Two other retinal degenerative disorders are glaucoma and diabetic retinopathy.

Other types of retinal degenerative disorders include retinopathy of prematurity, Usher syndrome (an inherited condition characterized by hearing loss and progressive loss of vision from RP), Stargardt's disease (inherited juvenile macular degeneration), Leber Congenital Amaurosis (an inherited disease characterized by loss of vision at birth), choroideremia (an inherited condition causing progressive vision loss due to degeneration of the choroid and retina), Bardet-Biedl syndrome (a complex of disorders that includes retinal degeneration and can also include polydactyly and renal disease), and Refsum disease (a disorder caused by inability to metabolize phytanic acid which is characterized by, inter alia, RP). See, e.g., Goodwin, Curr Opin Ophthalmol 19(3):255-62 (2008); Bonnet et al., Curr Opin Neural. 25(1):42-9 (2012); Coussa et al., Ophthalmic Genet. 33(2):57-65 (2012).

Other, rarer retinal degenerative disorders that may be treated or prevented using the methods and compositions described herein include Best's disease, cone-rod retinal dystrophy, gyrate atrophy, Oguchi disease, juvenile retinoschisis, Bassen-Kornzweig disease (abetalipoproteinemia), blue cone monochromatism disease, dominant drusen, Goldman-Favre vitreoretinal dystrophy (enhanced S-cone syndrome), Kearns-Sayre syndrome, Laurence-Moon syndrome, peripapillary choroidal dystrophy, pigment pattern dystrophy, (including Butterfly-shaped pigment dystrophy of the fovea, North Carolina macular dystrophy, macro-reticular dystrophy, spider dystrophy and Sjogren reticular pigment epithelium dystrophy), Sorsby macular dystrophy, Stickler's syndrome, and Wagner's syndrome (vitreoretinal dystrophy).

The retinal degenerative disorders that may be treated or prevented with the compositions and methods described herein can include the loss (e.g., death, e.g., apoptosis) of one or more types of retinal neurons. Compound I may be useful, e.g., to slow or prevent the loss of retinal neurons, thereby treating or preventing the retinal degenerative disorder.

Retinal Detachment and Retinal Trauma

Compound I may be useful in the treatment of trauma to the retina (retinal trauma), e.g., retinal detachment (e.g., that results from trauma to the retina), blunt trauma, chemical injury, physical injury, as a consequence of traumatic brain injury, or retinal trauma in a subject who has undergone surgery in or near the retina (surgical trauma, e.g., as a complication of a surgery such as anti-glaucomatous surgery).

Compound I may be useful in the treatment of retinal detachment, e.g., that is the result of trauma to the retina.

Compound I may be useful in the treatment of retinal detachment, e.g., that is not the result of trauma to the retina. For example, Compound I may be useful in the treatment of retinal detachment that results from shrinkage or contraction of the vitreous, advanced diabetes, an inflammatory eye disorder, severe myopia, retinal tears, family history, and complications from cataract surgery.

Compound I may be useful in the prevention of trauma to the retina (e.g., in a subject at risk for trauma to the retina, e.g., who will undergo surgery in or near the retina (surgical trauma, e.g., as a complication of a surgery such as anti-glaucomatous surgery).

Compound I may be useful in the prevention of retinal detachment (e.g., in a subject at risk for retinal detachment, e.g., who has a retinal tear or other risk factor (e.g., shrinkage or contraction of the vitreous, advanced diabetes, an inflammatory eye disorder, severe myopia, retinal tears, family history, and complications from cataract surgery)).

The retinal detachment that may be treated or prevented with the compositions and methods described herein can include the loss (e.g., death, e.g., apoptosis) of one or more types of retinal neurons. Compound I may be used, e.g., to slow or prevent the loss of retinal neurons, thereby treating or preventing the retinal detachment.

The retinal trauma that may be treated or prevented with the compositions and methods described herein can include the loss (e.g., death, e.g., apoptosis) of one or more types of retinal neurons. Compound I may be used, e.g., to slow or prevent the loss of retinal neurons, thereby treating or preventing the trauma to the retina.

Subjects at Risk of Developing a Retinal Disorder

Aspects of the present invention relate to administering Compound I to a subject who is not afflicted with a retinal disorder, as well as compositions for treating such subjects. For example, Compound I may be administered to a subject who is at risk of developing a retinal disorder. The risk factors described below are exemplary and are not intended to be limiting. In some embodiments, the dose administered to a subject at risk or developing a retinal disorder is lower than a dose which would be effective to treat a subject afflicted with the disorder. For example, the dose may be about 5, 10, 15, 20, 25, 30, 35, 40, 45, 50, 55, 60, 65, 70, 75, 80, 85, 90, 95, 3-25, 25-50, or 50-75% less than would be effective to treat a subject afflicted with the retinal disorder. In various embodiments, Compound I may be administered to a subject at risk of developing a retinal disorder than in a corresponding subject afflicted with the disorder.

In various embodiments, subjects who are at risk of developing a retinal disorder include subjects who have at least one grandparent, parent, aunt, uncle, sibling, and/or child who is afflicted with the disorder. In some embodiments, a subject who has one, two, three, or four grandparents, aunts or uncles, siblings, and/or children, and/or one or two parents with wet AMD, dry AMD, retinitis pigmentosa, Usher syndrome, Stargardt's disease, Leber Congenital Amaurosis, choroideremia, Bardet-Biedl syndrome, or Refsum disease, is at risk of developing the disorder.

In some embodiments a subject who has one, two, three, or four grandparents, aunts or uncles, siblings, and/or children and/or one or two parents with Best's disease, cone-rod retinal dystrophy, gyrate atrophy, Oguchi disease, juvenile retinoschisis, Bassen-Kornzweig disease (abetalipoproteinemia), blue cone monochromatism disease, dominant drusen, Goldman-Favre vitreoretinal dystrophy (enhanced S-cone syndrome), Kearns-Sayre syndrome, Laurence-Moon syndrome, peripapillary choroidal dystrophy, pigment pattern dystrophy, (including Butterfly-shaped pigment dystrophy of the fovea, North Carolina macular dystrophy, macro-reticular dystrophy, spider dystrophy and Sjogren reticular pigment epithelium dystrophy), Sorsby macular dystrophy, Stickler's syndrome, or Wagner's syndrome (vitreoretinal dystrophy) is at risk of developing that disorder.

In certain embodiments, subjects who are at risk of developing a retinal disorder include subjects having a genetic marker associated with the retinal disorder.

Additional risk factors are known for various diseases. For example, the following groups of subjects are at risk of developing AMD: subjects who are current and former smokers; subjects with high cholesterol (e.g. a level higher than about 100, 105, 110, 120, 125, 130, 130-159, 160-189, 190 milligrams (mg) of LDL cholesterol per deciliter (dL) of blood, or higher than about 200-239 or 240 mg of total cholesterol per dL of blood); subjects with hypertension; obese subjects; subjects who are at least 50, 55, 60, 65, 70, or 75 years old; self-identified Caucasian or white subjects; subjects having a genetic marker for AMD; female subjects; subjects with diets high in fat, cholesterol and sugar and low in antioxidants and green leafy vegetables; subjects with light (such as blue or green) colored eyes; subjects who have a parent, child, or sibling with macular degeneration; subjects with a variant of ARMS2/HTRA associated with AMD; subjects with a variant of complement associated with AMD; or subjects with an AMD-associated variant of a protein involved in cholesterol metabolism or collagen production. Subjects who fit into one, two, three, four, five, or more of these groups may be particularly at risk of developing AMD.

In some embodiments, a subject at risk of developing diabetic retinopathy is afflicted with diabetes (e.g. Type I or Type II diabetes).

In various embodiments, a subject at risk of retinal detachment is a subject who has experienced blunt trauma; a chemical injury (e.g., from exposure of the eyes to a toxic chemical); traumatic brain injury; or surgery in or near the retina.

Retinal Neurons

Compound I may be useful to increase the survival or maintain or increase the number of one or more types of retinal neurons (e.g., decrease the loss of one or more types of retinal neurons, e.g., decrease apoptosis of one or more types of retinal neurons, or increase the number of such cells).

The retina (neural portion of the eye) is part of the central nervous system. During development, the retina forms as an outpocketing of the diencephalon, called the optic vesicle, which undergoes invagination to form the optic cup. The inner wall of the optic cup gives rise to the retina, while the outer wall gives rise to the pigmented epithelium. This epithelium is a melanin-containing structure that reduces backscattering of light that enters the eye; it also plays a critical role in the maintenance of photoreceptors, renewing photopigments and phagocytosing the photoreceptor discs, whose turnover at a high rate is essential to vision.

The retina comprises complex neural circuitry that converts the graded electrical activity of photoreceptors into action potentials that travel to the brain via axons in the optic nerve. There are five types of retinal neurons (photoreceptors, bipolar cells, ganglion cells, horizontal cells, and amacrine cells) and one type of glia (Müller glia) in the retina. The cell bodies and processes of these neurons are stacked in five alternating layers, with the cell bodies located in the outer nuclear, inner nuclear and ganglion cell layers, and the processes and synaptic contacts located in the outer plexiform and inner plexiform layers. A direct three-neuron chain—photoreceptor cell to bipolar cell to ganglion cell—is the major route of information flow from photoreceptors to the optic nerve.

There are two types of light-sensitive photoreceptors in the retina: rods and cones. Both types of photoreceptors have an outer segment that is composed of membranous discs that contain photopigment and lies adjacent to the pigment epithelial layer, and an inner segment that contains the cell nucleus and gives rise to synaptic terminals that contact bipolar or horizontal cells. Absorption of light by the photopigment in the outer segment of the photoreceptors initiates a cascade of events that changes the membrane potential of the receptor, and therefore the amount of neurotransmitter released by the photoreceptor synapses onto the cells they contact. The synapses between photoreceptor terminals and bipolar cells (and horizontal cells) occur in the outer plexiform layer; more specifically, the cell bodies of photoreceptors make up the outer nuclear layer, whereas the cell bodies of bipolar cells lie in the inner nuclear layer. The short axonal processes of bipolar cells make synaptic contacts in turn on the dendritic processes of ganglion cells in the inner plexiform layer. The much larger axons of the ganglion cells form the optic nerve and carry information about retinal stimulation to the rest of the central nervous system.

The two other types of neurons in the retina, horizontal cells and amacrine cells, have their cell bodies in the inner nuclear layer and are primarily responsible for lateral interactions within the retina. These lateral interactions between receptors, horizontal cells, and bipolar cells in the outer plexiform layer are largely responsible for the visual system's sensitivity to luminance contrast over a wide range of light intensities. The processes of amacrine cells, which extend laterally in the inner plexiform layer, are postsynaptic to bipolar cell terminals and presynaptic to the dendrites of ganglion cells. The processes of horizontal cells ramify in the outer plexiform layer. Several subclasses of amacrine cells that make distinct contributions to visual function. One class of amacrine cells, for example, plays an important role in transforming the persistent responses of bipolar cells to light into the brief transient responses exhibited by some types of ganglion cells. Another type serves as an obligatory step in the pathway that transmits information from rod photoreceptors to retinal ganglion cells.

The outer segments of the photoreceptors contain membranous discs that house the light-sensitive photopigment and other proteins involved in the transduction process. These discs are formed near the inner segment of the photoreceptor and move toward the tip of the outer segment, where they are shed. The pigment epithelium plays an essential role in removing the expended receptor discs; all the discs in the outer segments are replaced every 12 days. In addition, the pigment epithelium contains the biochemical machinery that is required to regenerate photopigment molecules after they have been exposed to light. It is presumably the demands of the photoreceptor disc life cycle and photopigment recycling that explain why rods and cones are found in the outermost rather than the innermost layer of the retina. Disruptions in the normal relationships between pigment epithelium and retinal photoreceptors such as those that occur in retinitis pigmentosa have severe consequences for vision.

Another important cell type in the retina is the Müller glia. Müller glia span the entire thickness of the retina and function to support the neurons of the retina by maintaining homeostasis and retinal integrity. During injury or disease, Müller glia can have a positive or negative impact on retinal function. In lower vertebrates, such as teleost fish, Müller glia can respond to injury by reprogramming themselves to become progenitor cells capable of dividing and differentiating into new retinal neurons. In mammals, only under special circumstances can the Müller glia respond to generate new retinal neurons at a very low level (reviewed by Goldman (2014) Nature Reviews Neuroscience, 15, 431-442).

Compound I may be used to increase the survival of photoreceptors, bipolar cells, ganglion cells, horizontal cells, and/or amacrine cells.

Glial Cell Line Derived Neurotrophic Factor (GDNF)

Glial cell-derived neurotrophic factor, also known as GDNF, is a protein that, in humans, is encoded by the GDNF gene. GDNF is a small protein that potently promotes the survival of many types of neurons. GDNF is a highly conserved neurotrophic factor. The recombinant form of this protein was shown to promote the survival and differentiation of dopaminergic neurons in culture, and was able to prevent apoptosis of motor neurons induced by axotomy. The encoded protein is processed to a mature secreted form that exists as a homodimer. The mature form of the protein is a ligand for the product of the RET (rearranged during transfection) protooncogene. In addition to the transcript encoding GDNF, two additional alternative transcripts encoding distinct proteins, referred to as astrocyte-derived trophic factors, have also been described. Glial cell line-derived neurotrophic factor has been shown to interact with GFRA2 and GDNF family receptor alpha 1.

A feature of GDNF is its ability to support the survival of dopaminergic and motorneurons. These neuronal populations die in the course of Parkinson's disease and amyotrophic lateral sclerosis (ALS). GDNF also regulates kidney development and spermatogenesis, and it affects alcohol consumption.

GDNF has been shown to delay photoreceptor degeneration in a transgenic rat model of Retinitis Pigmentosa. See Sanftner et al., Molecular Therapy 4(6):622-629 (2001).

GDNF has been shown to protect retinal ganglion cells in a rat model of glaucoma (Jiang et al., Molec. Vision 13:1783-1792 (2007)); a DBA/2J (D2) mouse, which is a murine model of spontaneous glaucoma (Ward et al., J. Pharma. Sciences 96:558-568 (2007)); and a pig model of acute retinal ischemia (Voss Kyhn et al., Exper. Eye Res. 89:1012-1020 (2009).

An mRNA sequence of the human GDNF gene is Genbank Accession Number NM_000514 (version NM_000514.3 GI:299473777). (SEQ ID NO:2)

An amino acid sequence of human GDNF is GenPept Accession Number NP_000505 (version NP_000505.1 GI:4503975):

| (SEQ ID NO: 1) | |
|---|---|
| 1 | mklwdvvavc lvllhtasaf plpagkrppe apaedrslgr rrapfalssd snmpedypdq |
| 61 | fddvmdfiqa tikrlkrspd kqmavlprre rnrqaaaanp ensrgkgrrg qrgknrgcvl |
| 121 | taihlnvtdl glgyetkeel ifrycsgscd aaettydkil knlsrnrrlv sdkvgqaccr |
| 181 | piafdddlsf lddnlvyhil rkhsakrcgc i |

See also WO 2008/100966 (Example 8) and U.S. Patent Application No. 2009/0069230, published Mar. 12, 2009, the entire contents of each of which are hereby incorporated herein by reference.

A mouse mRNA GDNF sequence is Genbank Accession Number NM_010275 (version NM_010275.3 GI:672349279) (SEQ ID NO:3).

A mouse GDNF amino acid sequence is GenPept Accession Number NP_034405 (version NP_034405.1 GI:7110601) (SEQ ID NO:4).

A rat GDNF mRNA sequence is Genbank Accession Number NM_019139 (version NM_019139.1 GI:9506720) (SEQ ID NO:5).

A rat GDNF amino acid sequence is GenPept Accession Number NP_062012 (version NP_062012.1 GI:9506721) (SEQ ID NO:6).

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to 5.0 mg.

Pharmaceutical Formulations and Delivery

Dosages, formulations, dosage volumes, regimens, and methods for inducing GDNF protein expression can vary. Thus, minimum and maximum effective dosages vary depending on the method of administration. Suppression of the clinical and histological changes associated with a retinal degenerative disease can occur within a specific dosage range, which, however, varies depending on the organism (e.g., subject) receiving the dosage, the route of administration, whether Compound I (e.g., a pharmaceutical composition comprising Compound I) is administered in conjunction with another agent(s), and the specific regimen of Compound I administration. For example, in general, nasal administration requires a smaller dosage than oral or enteral administration.

In various embodiments of the invention, a composition comprising Compound I may be administered only once or multiple times. For example, Compound I may be administered using a method disclosed herein at least about once, twice, three times, four times, five times, six times, or seven times per day week, month, or year. In some embodiments, a composition comprising Compound I is administered once per month. In certain embodiments, the composition is administered once per month via intravitreal injection. In various embodiments, such as embodiments involving eye drops, a composition is self-administered.

For the treatment of a retinal degenerative disorder, Compound I (e.g., a pharmaceutical composition comprising Compound I) may be administered locally, e.g., as a topical eye drop, peri-ocular injection (e.g., sub-tenon), intraocular injection, intravitreal injection, retrobulbar injection, intraretinal injection, subconjunctival injection, or using iontophoresis, or peri-ocular devices which can actively or passively deliver drug.

Sustained release of drug may be achieved by the use of technologies such as implants (e.g., solid implants) (which may or may not be bio-degradable) or bio-degradable polymeric matrices (e.g., micro-particles). These may be administered, e.g., peri-ocularly or intravitreally.

Pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils.

For treatments of the eye or other external tissues, such as the mouth or skin, the formulations (e.g., a pharmaceutical composition comprising Compound I) may be applied as a topical ointment or cream. When formulated in an ointment, Compound I may be employed with either a paraffinic or a water-miscible ointment base. Alternatively, Compound I may be formulated in a cream with an oil-in-water cream base or a water-in-oil base.

Pharmaceutical formulations adapted for topical administrations to the eye include eye drops wherein Compound I is dissolved or suspended in a suitable carrier, especially an aqueous solvent. Formulations to be administered to the eye will have ophthalmically compatible pH and osmolality. The term “ophthalmically acceptable vehicle” means a pharmaceutical composition having physical properties (e.g., pH and/or osmolality) that are physiologically compatible with ophthalmic tissues.

In some embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In various embodiments, the ophthalmic formulations of the present invention may be in the form of liquid, solid or semisolid dosage form. The ophthalmic formulations of the present invention may comprise, depending on the final dosage form, suitable ophthalmically acceptable excipients. In some embodiments, the ophthalmic formulations are formulated to maintain a physiologically tolerable pH range. In certain embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In some embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

One or more ophthalmically acceptable pH adjusting agents and/or buffering agents can be included in a composition of the invention, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g., poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition, and polymeric coatings that will enhance drug diffusion, erosion, dissolution, and osmosis.

Formulations for drug delivery using ocular devices may combine one or more active agents and adjuvants appropriate for the indicated route of administration. For example, Compound I (optionally with another agent) may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters), and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3; 57:1595-639 (2005), the entire content of which is incorporated herein by reference.

Biocompatible implants for placement in the eye have been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; 6,699,493; and 8,293,210, the entire contents of each of which are incorporated herein by reference.

The implants may be monolithic, i.e. having the active agent (e.g., Compound I) or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including Compound I, may be distributed in a non-homogenous pattern in the matrix. For example, the implant may include a portion that has a greater concentration of Compound I relative to a second portion of the implant.

The intraocular implants disclosed herein may have a size of between about 5 um and about 2 mm, or between about 10 um and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 ug, more preferably about 500-1000 ug. For example, an implant may be about 500 ug, or about 1000 ug. For non-human subject, the dimensions and total weight of the implant(s) may be larger or smaller, depending on the type of subject. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques, and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of 0.5 u.m to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Microspheres for ocular delivery are described, for example, in U.S. Pat. Nos. 5,837,226; 5,731,005; 5,641,750; 7,354,574; and U.S. Pub. No. 2008-0131484, the entire contents of each of which are incorporated herein by reference.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. Nos. 4,704,295; 4, 556,552; 4,309,404; and 4,309,406, the entire contents of each of which are incorporated herein by reference.

Experimental Techniques and Animal Models of Retinal Degeneration

Techniques and models that can be used to evaluate the utility of Compound I in treating retinal degenerative disorders include the following.

Loss of retinal neurons can be evaluated, e.g., by evaluating cell count (e.g., photoreceptor cell count).

Electroretinography is a process in which an electrode is placed on the cornea, the eye is stimulated by a flash of light, and the electrical activity of the photoreceptor cells is measured by the electrode. Odom J V, Leys M, Weinstein G W. Clinical visual electrophysiology. In: Tasman W, Jaeger E A, eds. Duane's Ophthalmology. 15th ed. Philadelphia, Pa.: Lippincott Williams & Wilkins; 2009: chap 5; Baloh R W, Jen J. Neuro-ophthalmology. In: Goldman L, Schafer A I, eds. Cecil Medicine. 24th ed. Philadelphia, Pa.: Saunders Elsevier; 2011:chap 432; Cleary T S, Reichel E. Electrophysiology. In: Yanoff M, Duker J S, eds. Ophthalmology. 3rd ed. St. Louis, Mo.: Mosby Elsevier; 2008:chap 6.9. See also, e.g., Mohand-Said S, et al., Proc Natl Acad Sci USA. 95:8357-8362 (1998) and Jaissle et al., Invest. Ophthalmol. Vis. Sci. 42(2):506-513 (2001).

Another measure of photoreceptor function that can be measured by retinography is a peak of electrical activity between 0.05 and 50 Hz following systemic introduction of sodium azide, known as the azide response. See, e.g., Ando and Noell, Jpn. J. Physiol. 43(3):323-333 (1993).

The Royal College of Surgeons rat (“RCS rat”) is an animal model of inherited retinal degeneration, in which retinal degeneration results from defective RPE cells that are unable to phagocytose photoreceptor outer segments. See, e.g., D'Cruz et al., Human Molecular Genetics 9(4):645-651 (2000). Histologically, the retina of the RCS rat is characterized by abnormal accumulation of outer segment debris between the photoreceptor cell outer segment layer and the retinal pigmented epithelium. Accumulation occurs prior to, and concomitant with, the death of photoreceptor cells. RCS rats experience progressive postnatal loss of photoreceptor cells and attendant loss of vision.

A transgenic rat model (TgN S334ter-4) expressing a mutated rhodopsin gene in which a termination codon is present at residue 334 of the opsin transgene, resulting in a protein lacking the 15 carboxy-terminal amino acids, is available. The C terminus is involved in rhodopsin localization to the outer segments and its absence contributes to photoreceptor cell death by a caspase-3-dependent mechanism. Multiple mutations within the C terminus have been identified in patients with Retinitis Pigmentosa. Thus, using TgN S334ter-4 rats enables us to design and test therapies in an animal model with a disease similar to human RP. The retinas of heterozygous TgN S334ter-4 rats develop normally and have 8-10 rows of photoreceptor nuclei in the outer nuclear layer (ONL) at postnatal day (P) 15. The time course of degeneration occurs in two phases beginning at about P15. The first phase, between P15 and P60, is fast with the ONL degenerating to 2-3 rows of nuclei accompanied by a substantially reduced eletroretinographic response by P60. Beyond P60 a slower rate of ONL loss ensues. See, e.g., Sanftner et al., Molecular Therapy 4(6):622-629 (2001); Green et al., Invest. Ophthalmol. Vis. Sci. 41:1546-1553 (2000); and Liu et al., J. Neurosci. 19:4778-4785 (1999), the entire contents of each of which are hereby incorporated herein by reference.

The rhodopsin knockout (Rho−/−) mouse is a model for Retinitis Pigmentosa (RP), a heterogeneous group of hereditary disorders of the rod system that cause progressive retinal degeneration. Homozygous (Rho−/−) mice carry a replacement mutation in exon 2 of the rhodopsin gene, and show a complete absence of rhodopsin and do not build rod outer segments. See, e.g., Humphries et al., Nat Genet. 15:216-219 (1997) and Jaissle et al., Invest. Ophthalmol. Vis. Sci. 42(2):506-513 (2001), the entire contents of each of which are hereby incorporated herein by reference. Another rhodopsin knockout (carrying a different null mutation) is described in Lem et al., Proc Natl Acad Sci USA 96:736-741 (1999), the entire content of which is hereby incorporated herein by reference.

Combination Therapy

For the treatment and/or prevention of a retinal disorder, Compound I may be administered alone or in combination with one or more additional therapies (e.g., agents or procedures), e.g., that are used to treat and/or prevent a given retinal disorder.

For example, for the treatment and/or prevention of a retinal disorder, Compound I may be used in combination with one or more of the following protein therapies: basic Fibroblast Growth Factor (bFGF), nerve growth factor (NGF), ciliary neurotrophic factor (CNTF), pigment epithelium derived factor (PEDF), or brain-derived neurotrophic factor (BDNF). As another example, Compound I can be used in combination with gene therapy for a retinal disorder. For example, Compound I can be used in combination with gene therapy to correct mutations such as retinal pigment epithelium-specific protein 65 kDa (RPE65) mutations which can cause severe hereditary blindness resulting from both dysfunction and degeneration of photoreceptors (see, e.g., Maguire et al., Lancet 374(9701):1597-1605 (2009)).

For example, for the treatment and/or prevention of retinitis pigmentosa, Compound I can be used in combination with one or more of the following therapies: vitamin A/beta-carotene, docosahexaenoic acid (DHA), acetazolamide, a calcium channel blocker, lutein/zeaxanthin, or valproic acid.

As another example, for the treatment and/or prevention of wet AMD, Compound I can be used in combination with one or more of the following therapies: a laser treatment, such as laser photocoagulation or photodynamic therapy (e.g., with verteporfin (VISUDYNE™)), a VEGF Inhibitor, pegaptanib (e.g., MACUGEN®), Ranibizumab (e.g., LUCENTIS®), Bevacizumab (e.g., AVASTIN®), or aflibercept (e.g., EYLEA®).

As a further example, for the treatment and/or prevention of dry AMD, Compound I can be used in combination with one or more of the following therapies: vitamin supplementation, e.g., 500 milligrams (mg) of vitamin C, 400 international units (IU) of vitamin E, 15 mg of beta carotene (often as vitamin A—up to 25,000 IU), 80 mg of zinc (as zinc oxide), or 2 mg of copper (as cupric oxide).

As another example, for the treatment and/or prevention of glaucoma, Compound I can be used in combination with one or more of the following therapies: eyedrops, e.g., of a prostaglandin, a beta blocker, an alpha-adrenergic agonist, a carbonic anhydrase inhibitor, a miotic or cholinergic agent, or a combined medication (such as a beta blocker and alpha adrenergic agonist, or a beta blocker and carbonic anhydrase inhibitor); an oral medication, such as a carbonic anhydrase inhibitor; laser surgery, filtering surgery (trabeculectomy), or drainage implants.

As another example, for the treatment and/or prevention of diabetic retinopathy, Compound I can be used in combination with one or more of the following therapies: surgery to reattach the retina, focal laser treatment, scatter laser treatment, or vitrectomy.

For the treatment of retinal trauma, for example, Compound I can be used in combination with surgery to repair the trauma.

For the treatment of retinal detachment, for example, Compound I can be used in combination with one or more of the following therapies: surgery to reattach the retina, photocoagulation, cryopexy, pneumatic retinopexy, scleral buckling, or vitrectomy.

The term “combination” refers to the use of the two or more therapies to treat the same patient, wherein the use or actions of the therapies overlap in time. The therapies can be administered at the same time (e.g., as a single formulation that is administered to a patient or as two separate formulations administered concurrently) or sequentially in any order. Sequential administrations are administrations that are given at different times. The time between administration of the one therapy and another therapy can be minutes, hours, days, or weeks. Compound I may also be used to reduce the dosage of another therapy, e.g., to reduce the side-effects associated with another agent that is being administered (and vice versa). Accordingly, a combination can include administering a second agent at a dosage at least about 10, 20, 30, or 50% lower than would be used in the absence of Compound I (and vice versa).

Compound I and any other agents will typically be administered in the form of one or more pharmaceutical compositions, wherein the composition comprises, in addition to the active ingredient(s), one or more pharmaceutically acceptable carriers and/or excipients such as are known in the art. A composition (e.g., pharmaceutical composition) may include an effective amount of Compound I (alone or in combination with one or more other agents).

All publications, patent applications, patents, and other references mentioned herein are incorporated by reference in their entirety. In the case of conflict, the present specification, including definitions, controls. In addition, the materials, methods, and examples are illustrative only and not intended to be limiting.

### EXAMPLES

Retinal degenerative disorders, such as retinitis pigmentosa and age-related macular degeneration are characterized by irreversible loss of photoreceptors. Several growth factors, including GDNF, have been shown to rescue retinal neurons in animal models of retinal disease.

### Example 1: Compound I Preparation

Formulation of the Aqueous Solution of the Compound I Maleate Salt:

The vehicle (pH 6.0) of the aqueous Compound I maleate salt solution was prepared based on the following composition:

| | Concentration | Concentration |
|---|---|---|
| Component | (mg/ml) | (mM) |
| NaH₂PO₄•2H₂O | 1.37 | 8.8 |
| Na₂HPO4 | 0.17 | 1.2 |
| Mannitol | 50 | |
| Captisol ® | 50 | |
| WFI | Q.S. | Q.S. |

WFI = water for injection
Q.S = quantum satis- added to the designated total volume

The Compound I maleate salt was dissolved in the vehicle to make 0.5 mg/ml (in free base) solution.

Procedure to Convert Maleate Salt to Free Base:

The Compound I maleate salt (synthesized according to the procedure in WO 2006/125622) was suspended in acetonitrile:water (95:5) solvent (1 g in 10 ml) followed by the addition of 1.5-fold mol equivalent NaOH. The slurry was then mixed at room temperature for 24 hours followed by filtration through filter paper. The filter cake was rinsed with the 95:5 acetonitrile:water solvent for at least three times and dried at 65° C. for 24 hours. The structure of the free base was confirmed by NMR.

Formulation of 60 mg/mL Suspension of Compound I Free Base:

The vehicle (pH 7.2) of the Compound I free base suspension was prepared based on the following composition:

| | Concentration | Concentration |
|---|---|---|
| Component | (mg/ml) | (mM) |
| Lutrol 68 | 4 | |
| NaH₂PO₄•2H₂O | 0.62 | 4 |
| Na₂HPO4 | 0.85 | 6 |
| Mannitol | 45 | |
| PEG3350 | 20 | |
| WFI | Q.S. | Q.S. |

The Compound I free base was suspended in the vehicle to make a 60 mg/ml uniform, white suspension by vortexing.

### Example 2: Compound I Induces GDNF Expression and Increases the Survival and Number of Photoreceptor Cells

Degenerative diseases of the retina, such as glaucoma, retinitis pigmentosa and age-related macular degeneration are characterized by the irreversible loss of retinal neurons. Several growth factors, including glial cell derived neurotrophic factor (GDNF), have been shown to rescue retinal neurons in animal models of retinal disease. GDNF induction has been observed in normal and diseased retina by amitriptyline. This non-limiting example presents a study of the surprising ability of a small molecule (Compound I), which was found in a GDNF induction phenotypic screen in ES-derived astrocytes and C6 glioma cell line, to induce GDNF in vitro/in vivo and rescue photoreceptors.

GDNF induction in vitro was assessed in three art recognized models for human retinal disorders: human ARPE19, human retinal progenitor cells (hRPCs) and mouse pluripotent cell-derived eyecups. For time-course pharmacokinetic and GDNF induction studies in C57Bl/6 mice, Compound I sustained release formulation was injected intravitreally. The same delivery approach was used in the rhodopsin knockout mouse model of retinal degeneration (rho−/−) mice to assess long term GDNF induction and photoreceptor rescue.

The suspension provided sustained Compound I delivery with 28 ug of drug remaining in the eye 2 weeks after a single injection. Compound I suspension injection in C57Bl/6 mice resulted in significant upregulation of GDNF mRNA (>1.8 fold) and protein levels (>2.8 fold). Importantly, Compound I treatment resulted in outer nuclear layer preservation in rho−/− mice with a 2-fold increase in photoreceptor number compared to control.

Compound I was found to function as a potent neuroprotective compound that induces expression of GDNF in normal and diseased retina. This induction results in photoreceptor rescue in subjects with disorders characterized by loss of retinal neuronal cells.

Conditions Characterized by Loss of Retinal Neuronal Cells

Retinal degenerative disorders, such as glaucoma, retinitis pigmentosa and age-related macular degeneration are characterized by irreversible loss of photoreceptors. Several therapeutic strategies are being explored to delay or substitute for such loss: neuroprotection (CNTF—Pilli et al., Retina 34(7):1384-1390 (2014)); gene correction (RPE65—Maguire et al., Lancet 374(9701):1597-1605 (2009)); cell replacement (RPE—Schwartz et al., Lancet 379(9817):713-720 (2012)); optogenetics (Lagali et al., Nat. Neurosci. 1, 667-675(2008)); or photosensitive chip implantation (ARGUS—da Cruz et al., Br J Ophthalmol. 97(5):632-636 (2013)). One benefit of neuroprotection is that it can be applied to many neurodegenerative diseases at early stages, while cell and tissue structure is still preserved.

One approach to neuroprotection is the use of growth factors and small molecules. Several of these compounds, such as ciliary neurotrophic factor (CNTFX) (Chew et al., Am. J. Ophthalmol. 159, 659-666.e1 (2015)), brain-derived neurotrophic factor (BDNF), nerve growth factor (NGF) (Huo et al., Curr. Eye Res. 37, 749-758 (2012)), pigment epithelium derived factor (PEDF) (Wang et al., ASN Neuro (2013)), glial-derived neurotrophic factor (GDNF), Vitamin A (Radu et al., Invest. Ophthalmol. Vis. Sci. 49, 3821-3829 (2008)), Docosahexaenoic acid (Mirza et al., PLoS ONE 8, e75963 (2013)), Lutein (Woo et al., Graefes Arch. Clin. Exp. Ophthalmol. 251, 41-51 (2013)), valproic acid (Mitton et al., Mol. Vis. 20, 1527-1544 (2014)) and rasagiline (Eigeldinger-Berthou et al., Retina (Philadelphia, Pa.) 32, 617-628 (2012)) have been shown to rescue retinal structure in various animal models of retinal disease, although none of them have yet emerged as a viable therapeutic option. This is partially due to limited delivery options of growth factors: they can be administered as recombinant proteins, overexpressed in host cells by genetic vectors or delivered by transplantation of overexpressing cells. In addition to the high cost of these approaches, the ability to control the level of the growth factor bioavailability and its delivery to targeted cells is limited. An alternative neuroprotection therapy approach was explored, where glial cell-derived growth factor (GDNF) is induced in host cells by small molecules.

Indirect neuroprotection is an emerging alternative. With a growing knowledge of growth factor induction and release, one can manipulate the production of growth factors in host cells using small molecule triggers. This approach has been extensively studied for GDNF production due to its importance in central nervous system development and repair. For example, it has been shown that GDNF may be induced by various classes of molecules (Saavedra et al., Prog. Neurobiol. 86, 186-215 (2008)) such as Amitriptyline (Hisaoka et al., J. Neurochem. 79, 25-34 (2001)) (tricyclic antidepressant) and Valproic acid (Mitton et al., Mol. Vis. 20, 1527-1544 (2014)) (VPA, histone deacetylase inhibitor). Systemic treatment with VPA resulted in partial photoreceptor rescue in the rd10 mouse model of photoreceptor degeneration (Mitton et al., Mol. Vis. 20, 1527-1544 (2014)).

However, previous clinical studies showed no significant improvement in vision, following administration (Bhalla et al., Br J Ophthalmol 97, 895-899 (2013)). The use of these small molecules is limited due to their known pharmacokinetic properties after systemic application. For example, the effective concentrations identified for these compounds in vitro are far above 10 micromolar, which is problematic for clinical application.

Compound I

Compound I was previously identified in a GDNF induction phenotypic screen in ES-derived astrocytes and rat C6 glioma cell line by GlaxoSmithKline (GSK). Compound I has polypharmacology antagonizing multiple receptors including dopamine 2 and dopamine 3 receptors and for serotonin 2A, 2C and 5HT6 receptors (data not shown). This molecule induces GDNF at concentrations below 50 nM in mouse iPS-derived eyecups. That single intravitreal injection of Compound I was observed to lead to GDNF upregulation at the mRNA and protein level in both healthy and diseased mouse retinas. A sustained released formulation (suspension) was developed in order to achieve sustained delivery after a single injection. With this approach micrograms of the compound remained in the eye for at least two weeks after intravitreal delivery as shown by pharmacokinetic analysis. A single intravitreal injection of this sustained release formulation of Compound I led to sufficient GDNF induction to rescue photoreceptors in a severe retinal degeneration model, rhodopsin knockout mice (rho−/−).

These results indicate that Compound I is a potent neuroprotectant, with the effect at least partially mediated by GDNF induction. This study indicates that administering Compound I is a viable therapeutic strategy for retinal degenerative disorders and growth factor induction in the retina, e.g., to treat diseases such as retinitis pigmentosa.

Materials and Methods

The experiments outlined below were designed to investigate: 1) GDNF induction by Compound I in retinal specific in vitro assays; 2) GDNF induction by Compound I in normal and diseased mouse retina; 3) long-term effect of Compound I on the retina in a mouse model of retinitis pigmentosa.

Formulation of Compound I Solutions and Suspensions.

For Compound I solutions, the drug was dissolved in a vehicle consisting of 10 mM sodium phosphate buffer (pH 6.0), 50 mg/ml mannitol, 50 mg/ml captisol at a final stock concentration of 1 mM. The stock solution was then filtered through 0.45 μm filters and subsequently diluted with the vehicle into desired concentrations of 100, 30 and 10 μM. For Compound I suspensions (sustained release), the drug was suspended in a vehicle containing 4 mg/ml poloxamer 188, 20 mg/ml PEG 3350 and 45 mg/ml mannitol at 60 mg/ml by vortexing the drug-vehicle blend to form a whitish uniform suspension. The particle size of the suspension was measured to be D₁₀=6.8 μm; D₅₀=14.2 μm; D₉₀=26.5 μm.

GDNF Induction Assays.

GDNF induction in vivo was assessed using three retinal-specific cell systems: ARPE19, as a model for human retinal pigment epithelium; human retinal progenitor cells (hRPC), as a substitute for human retinal neurons/glia; and mouse eyecups, differentiated from induced pluripotent stem cells. ARPE19 cells are described in Dunn et al., Exp. Eye Res. 62: 155-169, 1996. To study GDNF induction in a human retinal pigment epithelium cell line, ARPE19, thawed ARPE19 cells (passage 33, ATCC) were plated on fibronectin (Akron)-coated plates (Nunc) in stimulation medium at a density of 10 k cells/sq.cm. The flasks were incubated at standard conditions: 37° C., 5% CO₂, 100% humidity. Cells were plated 24 hours prior to Compound I treatment and were grown in Ultraculture medium (Lonza), supplemented with 1×MEM NEAA (Gibco), 1× L-glutamine (Gibco), 1× Antibiotic/antimycotic (Gibco), 2 ng/ml bFGF (Peprotech), 1 ng/ml EGF (Peprotech). For qPCR analysis, the experiments were performed in 35 mm Petri dishes in 2 ml of medium with Compound I concentration of 100 nM (DMSO-based solution). Total mRNA was isolated at 0, 15, 30, 60, 120, 240 minutes post stimulation. For protein expression determination by ELISA, the experiments were performed in 96 well plates in 100 ul of medium with each condition (concentrations ranging from 0 to 10 uM) reproduced in 3 wells. For ELISA analysis, plates were removed from out of incubator 24 hours post stimulation and freeze-thawed followed by CHAPS lysis buffer treatment for total mRNA extraction. The ELISA analysis for GDNF was performed using GDNF DuoSet kit (R&D systems) using manufacturer's instructions.

Experiments with human retinal progenitor cell line (hRPC) were performed in a similar manner (Baranov et al., Tissue Eng Part A 20, 140124133442001-1475 (2014)): hRPC were thawed and plated (passage 15) in 96 well plates using the same conditions and density as described above for ARPE19. The cell treatment, sample collection and readout were performed using the same protocol. The work with human fetal cells was reviewed by the MEEI/SERI Institutional Review Board and found to be an exemption.

To study GDNF induction in retina-like tissue structures, mouse eyecups were differentiated from wild-type mouse induced pluripotent stem cells according to the protocol described previously (Eiraku, M. & Sasai, Y., Nat Protoc 7, 69-79 (2012)). At day 26 of differentiation, differentiation was confirmed by Crx-expression, and eyecups were cut to about 0.5 mm² in size and replated as a suspension in a 96-well format in Neurobasal media (Gibco), supplemented with 1× L-glutamine (Gibco) and 1×N₂ supplement (Gibco). Samples were treated 24 hours later with Compound I in concentrations ranging from 0 to 10 uM. The sample collection and ELISA were performed in the same way as for ARPE19 and hRPC. All ELISA experiments were repeated twice. The results of dose-dependent GDNF induction were analyzed by ANOVA (p<0.05).

GDNF Induction by Compound I Aqueous Solution in Wild-Type and Rhodopsin Knockout Mice.

All animal procedures were performed under general (intraperitoneal ketamine/xylazine) and topical (proparacaine drops) anesthesia. Tropicamide was applied for pupil dilation. After injection, the eye was treated with antibiotic ointment (Bacitracin-Neomycin-Polymyxin). The same procedures were applied to control eyes.

To study GDNF induction by Compound I aqueous solution, 1 ul of 10 uM (n=5) or 100 uM (n=5) water-based Compound I or 1 ul of Vehicle (n=5) was administered intravitreally using beveled glass needle, connected to a Hamilton syringe through polyethylene tubing. During the injection, corneal paracenthesis was performed to reduce intraocular pressure. Compound I was injected into 4-week old wildtype (C57/Bl6, Jackson labs) and rhodopsin knockout (Humphries et al., Nat. Genet. 15, 216-219 (1997)).

Three hours after the injection animals were euthanized using CO₂, then the death was confirmed by cervical dislocation. The treated and untreated eyes were enucleated and placed in 250 ul of chilled RNAlater buffer (Qiagen) and stored at 4° C. Later, eyes were excised and total neural retina with retinal pigment epithelium were collected into 400 ul of Lysis buffer (Qiagen RNeasy kit) with 5 mM beta-mercaptoethanol. Total RNA was isolated using RNeasy kit (Qiagen) and eluted with 100 ul of Ultrapure water (Life technologies). All flow through from RNA isolation (˜2.4 ml) was collected and mixed with 10 ml of ice-cold (−20° C.) acetone for protein precipitation. The protein was precipitated at −20° C. for 30 minutes, then pelleted at 3500 rpm for 20 min. Pellets were washed once with 100% ethanol, then reconstituted with 300 ul of RIPA buffer with 0.2 mM 4-benzenesulfonyl fluoride hydrochloride (Tocris). mRNA and protein expression were assessed by qPCR and Western Blot and normalized to GAPDH and βActin. The statistical analysis was performed using a T-test (p<0.05) with Bonferroni post-hoc test.

Pharmacokinetic Studies of Compound I Sustained Release Formulation (Suspension).

To profile pharmacokinetics of Compound I after single intravitreal injection, 2 ul of Compound I suspension (60 mg/ml) were administered intravitreally to 3 week old wild-type (C57/Bl6) mice in the same way as described above for the solution. A beveled glass needle connected to a Hamilton syringe through polyethylene tubing was used. During the injection, corneal paracenthesis was performed to reduce intraocular pressure.

For enucleation animals were euthanized at several timepoints after injection: 1 hour (n=5), 1 day (n=3), 2 days (n=3), 7 days (n=3) and 14 days (n=3). The eyes were enucleated and placed in chilled phosphate-buffered saline and kept on ice. The eyes were dissected within 30 minutes post-enucleation: total retina and vitreous were collected into pre-weighed tubes, weighted and snap frozen. For Compound I content in the eye analysis retina and vitreous humor samples were diluted in 0.1% formic acid in 90:10 (v:v) acetonitrile:water before being analyzed for Compound I using an analytical method based on protein precipitation, followed by HPLC/MS/MS analysis. Using a 25 μL aliquot of sample, the lower limit of quantification (LLQ) for Compound I was 1.00 ng/mL. The computer systems that were used on this study to acquire and quantify data included Analyst Version 1.6.1 and SMS2000 version 2.3. The results were analyzed by ANOVA (p<0.05).

GDNF Induction by Compound I Suspension in Wild-Type Mice.

To study GDNF induction by Compound I suspension (60 mg/ml), 2 ul of suspension (n=35) or Vehicle (n=5) were administered intravitreally to 3-week old wild-type (C57/Bl6) mice. A beveled glass needle connected to a Hamilton syringe through polyethylene tubing was ysed. During the injection, corneal paracenthesis was performed to reduce intraocular pressure.

Animals were euthanized at several timepoints after injection for enucleation and RNA/protein analysis (n=5 for each timepoint, n=4 for 7 days and 14 days): vehicle at 1 hour, Compound I at 1 hour, 1 day, 2 days, 7 days and 14 days. The treated and untreated eyes were enucleated and were collected for mRNA and protein expression as previously described. The treated and untreated eyes were enucleated and placed in 250 ul of chilled RNAlater buffer (Qiagen) and stored at 4° C. The eyes were excised and total neural retina with retinal pigment epithelium was collected into 400 ul of Lysis buffer (Qiagen RNeasy kit) with beta-mercaptoethanol. Total RNA was isolated using RNeasy kit (Qiagen) and eluted with 100 ul of Ultrapure water (Life technologies). All flow through from RNA isolation (˜2.4 ml) was collected and mixed with 10 ml of ice-cold (−20° C.) acetone for protein precipitation. The protein was precipitated at −20° C. for 30 minutes, then pelleted at 3500 rpm for 20 min. Pellets were washed once with 100% ethanol, then reconstituted with 300 ul of RIPA buffer with AEBSF. mRNA and protein expression were assessed by qPCR and Western blot (WB). The results were analyzed by ANOVA (p<0.05).

Photoreceptor Rescue by Compound I Suspension in Rhodopsin Knockout Mice.

To study photoreceptor rescue by Compound I suspension (60 mg/ml), 2 ul of suspension (n=5) or Vehicle (n=5) were administered intravitreally to 4-week old rhodopsin knockout mice using a beveled glass needle, connected to a Hamilton syringe through polyethylene tubing. During the injection, corneal paracenthesis was performed to reduce intraocular pressure. 10 weeks after the injection, eyes were enucleated and fixed in Davidson solution for histological processing: paraffin embedding and sectioning. Six micron thick sections were cut in the area close to optic nerve head (ONH) and stained with hematoxylin-eosin (H&E) or processed for immunohistochemical analysis (IHC). H&E-stained sections were imaged (Olympus imaging system DP72) and total photoreceptor number was estimated based on the nuclei count in the outer nuclear layer (ONL). Cells were counted in each 100 um retina span starting from optic nerve head on three sections for each eye (five eyes per group). Total photoreceptor number per section was also calculated.

For IHC, sections were deparaffinized, heated (95° C.) in citrate buffer (pH 6.0) for 30 minutes, then blocked and stained with the antibodies for the following antigens. The photoreceptors: Opsin Blue (Millipore), Cone Arrestin (Millipore), Opsin Red/Green (Millipore), Recoverin (Millipore), Rod Outer Membrane 1 (Abnova); Müller glia and astrocytes: Lh×2 (Millipore), Glutamate synthase (Abcam), Glial Fibrillar Acidic Protein (Millipore); bipolar cells: Protein Kinase C alpha (Santa Cruz); ganglion cells: NeuN (Millipore) and GDNF (Santa Cruz).

GDNF Induction In Vitro.

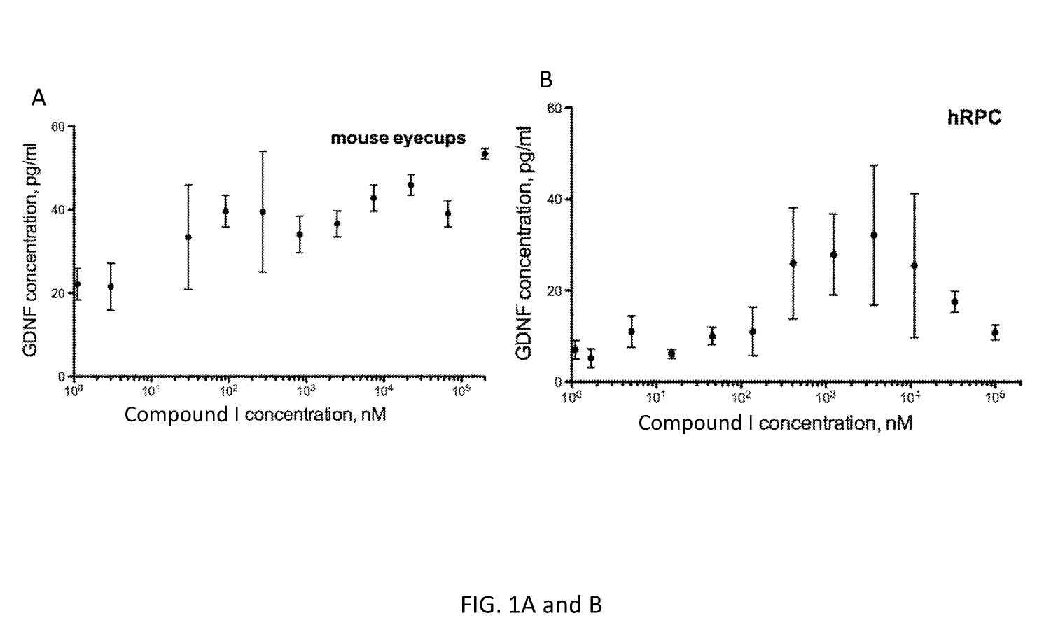

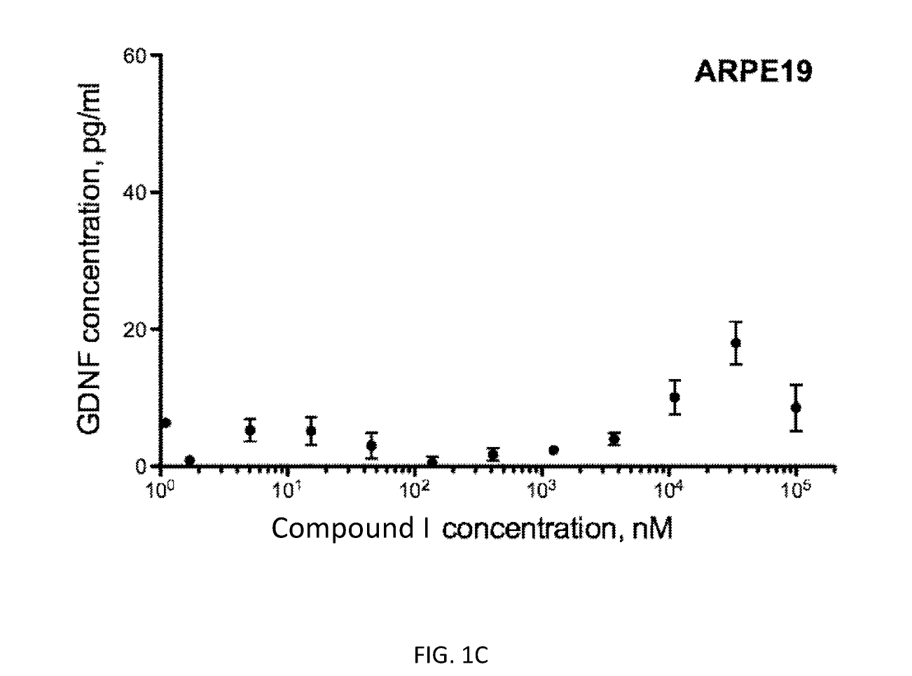

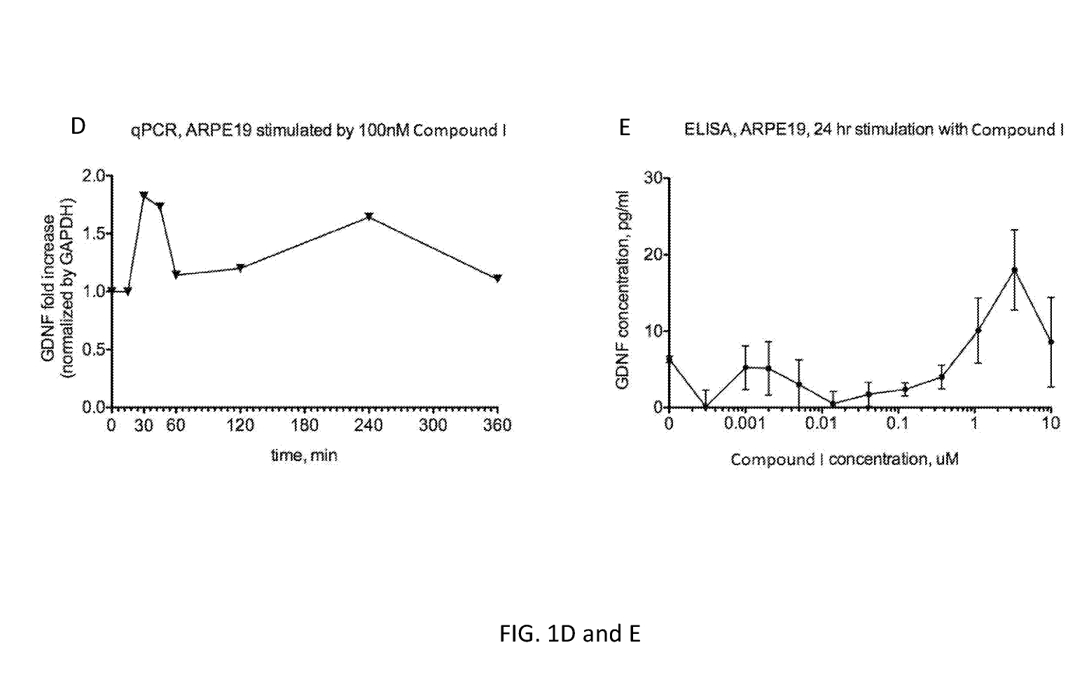

A goal was to determine if GDNF induction could be achieved by means of a small molecule, Compound I, in diseased retina. The first step was to determine if Compound I could induce GDNF in several in vitro models of the retina: mouse eye cups, human retinal progenitor cells, and the ARPE19 cell line. Human retinal pigmented epithelial cells (ARPE19) are an in vitro model of pigmented epithelial cells. Significant dose-dependent GDNF induction by Compound I was observed in all cell culture systems tested. Mouse eyecups, differentiated from induced pluripotent cells, showed the highest sensitivity: the upregulation of GDNF protein was observed at 30 nM concentration of Compound I (FIG. 1A). This system also showed the highest concentration of GDNF detected—up to 55 pg/ml. However it was also characterized by higher baseline level—around 20 pg/ml, compared to 5-10 pg/ml in adherent human cell lines tested. The human retinal progenitor cells (hRPC, FIG. 1B) and ARPE19 (FIG. 1C) upregulated GDNF after Compound I treatment at 1 and 3 uM, respectively. Compound I DMSO-based solution treatment of ARPE19 cells resulted in significant induction of GDNF on both mRNA and protein levels. The time-course of GDNF induction (normalized by GAPDH and ßActin) by Compound I in ARPE19 showed that GDNF mRNA peaked at 30 minutes after stimulation and then declined (FIG. 1D). GDNF protein (FIG. 1E) was upregulated at high concentrations of Compound I (1, 3 and 10 uM).

These results indicate that Compound I was able to induce GDNF in multiple in vitro models of the retina.

GDNF Induction by Compound I Aqueous Solution in Wild-Type and Rhodopsin Knockout Mice.

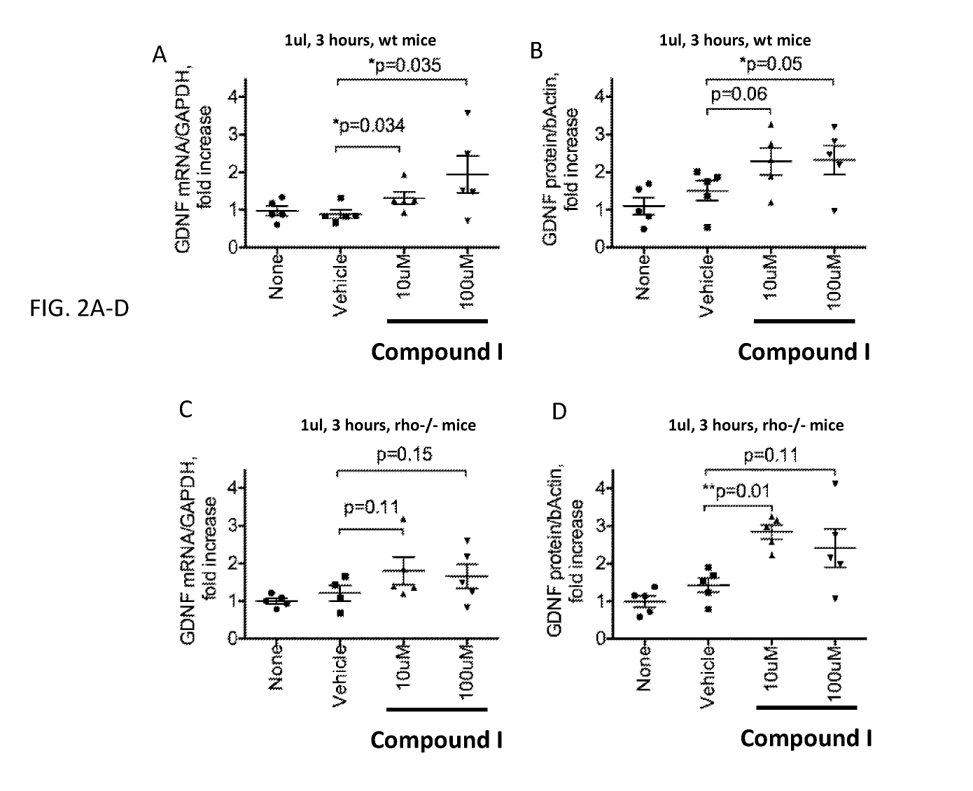

Since Compound I was an excellent inducer of GDNF expression in ARPE19 cells, we next tested to see if Compound I would induce GDNF mRNA and protein in wildtype and rhodophsin knockout mouse using a solution of Compound I. Significant induction of GDNF by 10 uM and 100 uM Compound I solution was observed on mRNA in wild type mice (FIG. 2A) (1.3 (p=0.03) and 1.9 (p=0.04) fold compared to vehicle-treated control, respectively) and in rhodopsin knockout mice (FIG. 2C) (1.8, p=0.11, 1.7, p=0.15). Protein was upregulated in vehicle-treated (1.5 fold) and Compound I-treated eyes (2.3 fold, both), compared to untreated control (FIG. 2B) in wildtype animals. In rhodopsin knockout mice protein was upregulated in vehicle-treated (1.2 fold) and Compound I-treated eyes (2.8 and 2.6 for 10 uM and 100 uM, respectively) (FIG. 2D).

These results show that Compound I was able to induce GDNF mRNA and protein in wild type and rhodopsin knockout mice.

Compound I Concentration after Compound I Suspension Injection in Wild-Type Mice.

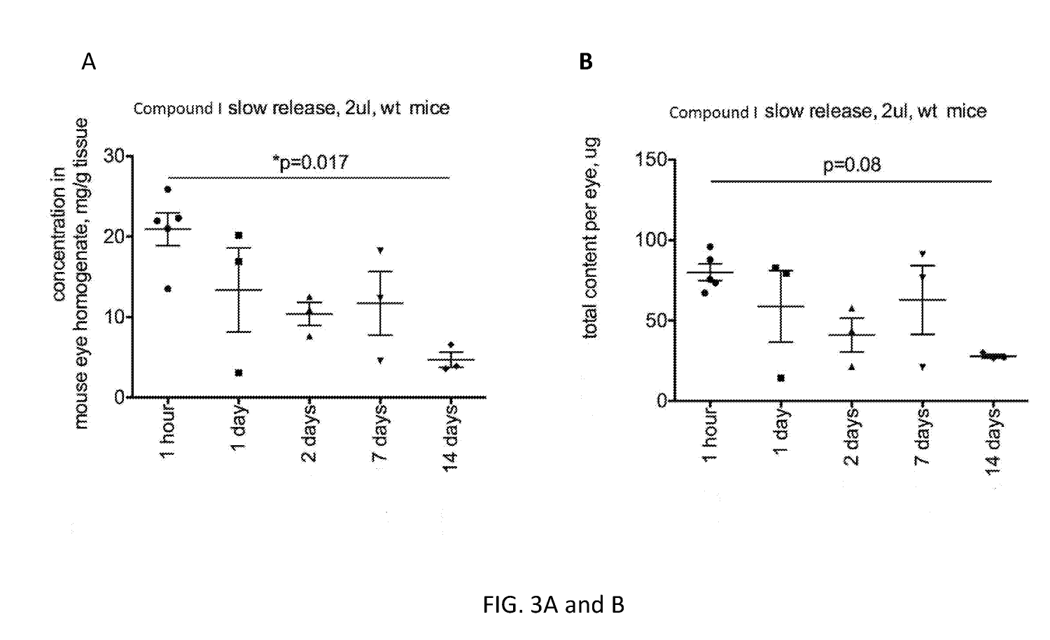

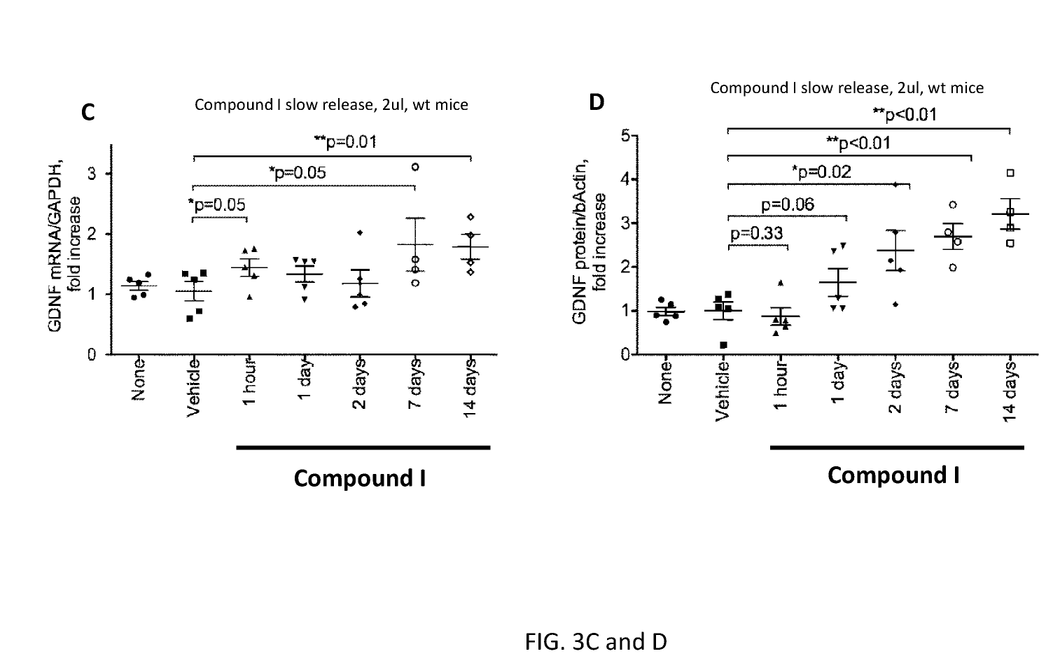

The next aim was to determine if Compound I, when given as a slow release formulation (suspension), would result in sustained release of Compound I, and therefore sustained induction of GDNF. Compound I can be reproducibly delivered intravitreally as suspension (FIG. 3A). To test if Compound I drug substance would last over a period of time via a single intravitreal injection as a suspension, wild type mice were injected and samples were collected at different time points over 14 days. A decline in Compound I depot size was observed during the course of the study (ANOVA p=0.017) but drug was still present after 2 weeks. The Compound I content in the collected vitreoretinal samples decreased from 21 ug to 5 ug per milligram of tissue. It corresponds to 80 ug at 1 hour post-injection to 28 ug (14 days post injection, FIG. 3A, B) of total Compound I amount. The presence of Compound I after 14 days shows that it is possible to utilize this formulation to produce long-term induction of GDNF.

GDNF Induction by Compound I Suspension in Wild-Type Mice.

A rationale for developing a suspension formulation of Compound I was to determine if GDNF could be induced in the retina at a sustained, but modest level, by a small molecule. Further characterization of the pharmocodynamics of the Compound I slow release formulation (suspension) was performed at 10 and 100 uM Compound I suspension. Compound I suspension treatment showed GDNF induction (FIG. 3C, D), similar to 10 uM Compound I aqueous solution (FIG. 2A, B). The GDNF mRNA remained upregulated at all timepoints (1.4, 1.3, 1.2, 1.8, 1.8 at 1 hour, 1 day, 2 days, 7 days and 14 days, respectively) (FIG. 3C). The protein level increased over time (FIG. 3D) (0.9, 1.6, 2.4, 2.7 and 3.2 at 1 hour, 1 day, 2 days, 7 days and 14 days, respectively). The achieved level of GDNF induction by the sustained release formulation exceeded levels observed in previous studies with a single Compound I injection.

Photoreceptor Rescue by Compound I Suspension in Rhodopsin Knockout Mice.

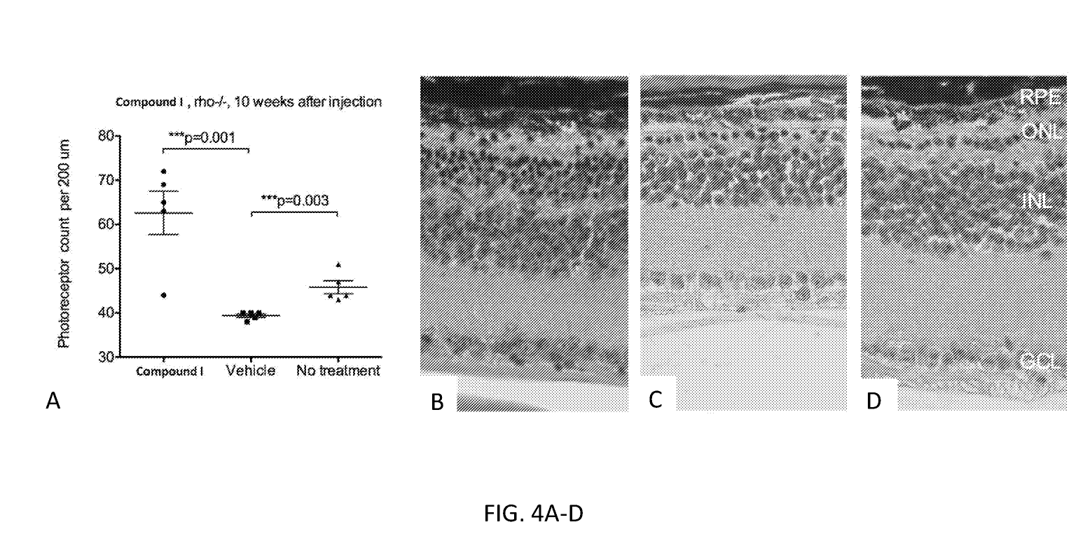

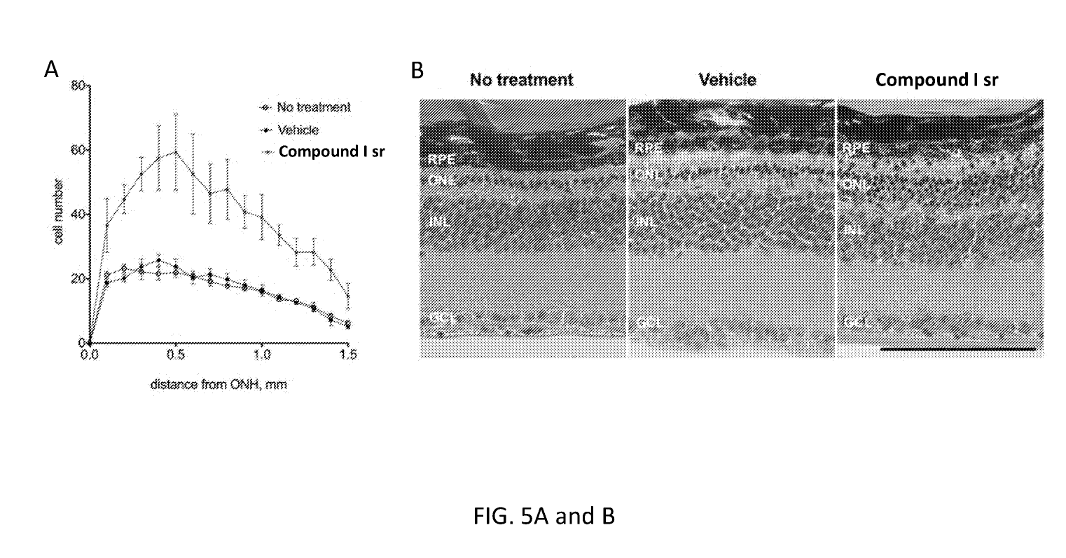

Finally, experiments were performed to test whether the GDNF induction achieved by Compound I suspension was capable to prevent near complete loss of the photoreceptors in the rhodopsin knockout mouse. The rhodopsin knockout mouse has photoreceptor degeneration that models the human disease of retinitis pigmentosa. This animal model is characterized by moderate progression of photoreceptor loss, starting at 4 weeks of age with only single layer of photoreceptors (predominantly cones) remaining by 15 weeks of age (Wang et al., Invest. Ophthalmol. Vis. Sci. 53, 3967-3972 (2012)). It was hypothesized that if Compound I suspension formulation was injected prior to the massive degeneration of the cones at week 3, photoreceptors may be maintained by the induction of GDNF through Compound I. Significant rescue of host photoreceptor layer in the rhodopsin knockout mice following Compound I treatment (FIG. 4B, C, D). The treatment resulted in 2.1 fold increase in photoreceptor cell number pan-retinally compared to uninjected and vehicle (1210 vs. 516 vs. 508 total photoreceptor cells per section, respectively) with significant retinal rescue of the photoreceptors layer throughout all retinal regions (FIG. 5A,B). The treatment resulted in 1.5 fold increase in photoreceptor cell number in the area close to the optic nerve head (ONH) (FIG. 4A) (63 vs 46 vs 39 photoreceptors per 200 um, Compound I vs Non-treated vs Vehicle control).

Next immunohistochemistry was performed to look for expression of proteins for photoreceptors Opsin blue (blue cone photoreceptors), Cone arrestin (pan cone), Opsin Opsin Red/Green (long wavelength cone photoreceptors), Recoverin (rod and cone photoreceptors) Rod Outer Membrane 1; Müller glia and astrocytes: Lh×2, Glutamate synthase, Glial Fibrillar Acidic Protein; bipolar cells: Protein Kinase C alpha; ganglion cells: NeuN (Millipore) and GDNF (data not shown). “Ectopic” photoreceptor-like cells were observed, expressing opsin blue in inner nuclear layer (INL), inner plexiform layer and ganglion cell layer in 4 out of 5 animals of the treatment group (data not shown). Other photoreceptor, glial, bipolar and ganglion cell markers were expressed in all groups (data not shown).

Ectopic photoreceptors after Compound I suspension treatment (data not shown)—Ectopic opsin blue—expressing cells were identified in 4 out of 5 Compound I-treated animals. None of those were observed in vehicle-treated animals. Cone Arrestin, or Lh×2 expression was similar among the groups. GDNF was expressed in photoreceptors in all of the groups.

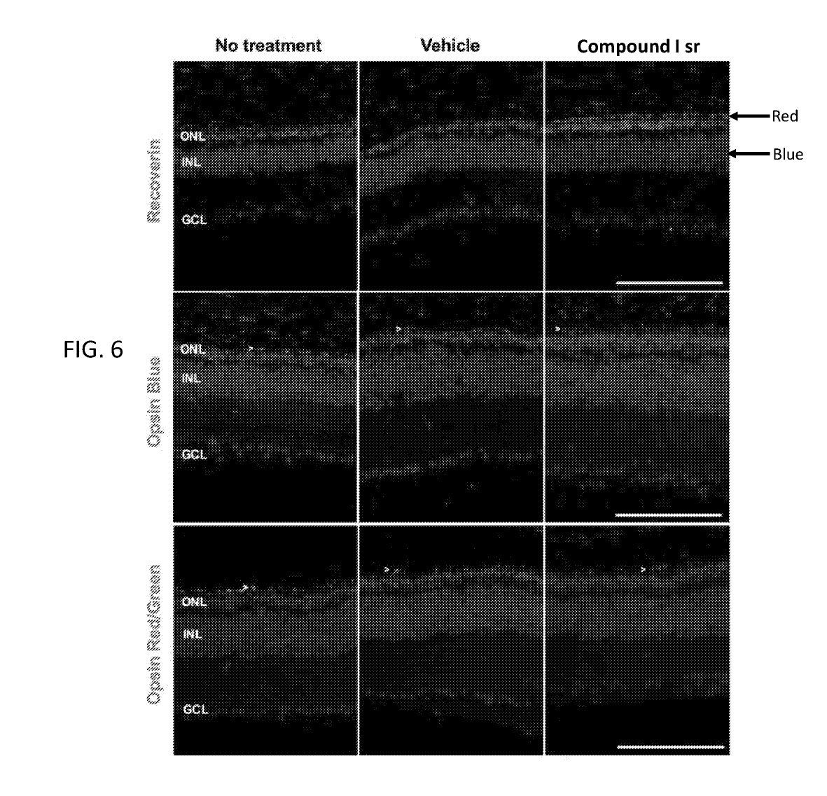

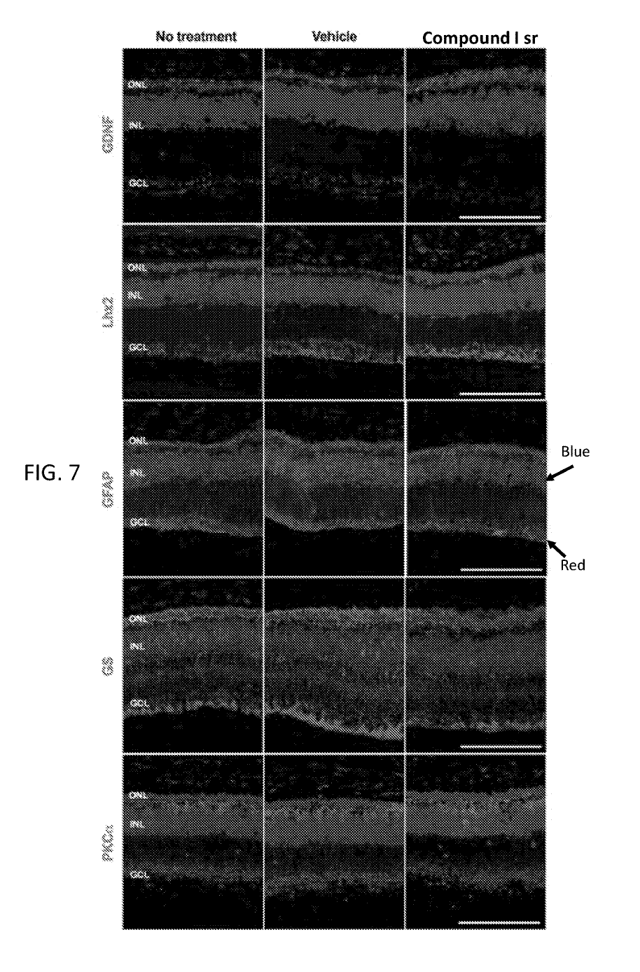

Significant difference in photoreceptor structure (outer segments) was not observed, however the signal was more intense in treated group, perhaps due to higher cell number in total (FIG. 6). Muller glia (Lh×2, GFAP, GS) and bipolar cell (PKCa) markers expression was similar between groups (FIG. 7). GDNF expression was confirmed in the outer nuclear level (ONL) (FIG. 7). Therefore cell counts, H&E staining, and immunostaining have shown that Compound I suspension is capable of rescuing a significant number of photoreceptors in the rhodopsin knock out mouse.

Compound I for Treatment of Retinal Neurodegenerative Disorders

Any significant delay of photoreceptor degeneration in these diseases will positively impact quality of life for many decades. The application of the present invention are extensive and do not require a full understanding of the genetic background of the disorders it is useful for treating or preventing.

Several approaches of neuroprotection have been tested with at least 6 classes of growth factors showing promise in various animal models of retinal degeneration and photoreceptor loss: Ciliary Neurotrophic Factor (CNTF) (Wen et al., Prog Retin Eye Res 31, 136-151 (2012)), Nerve Growth Factor (NGF) (Sun et al., Curr. Eye Res. 32, 765-772 (2007)), Brain Derived Neurotrophic Factor (BDNF) (Caffé et al., Ophthalmol. Vis. Sci. 42, 275-282 (2001)), basic Fibroblast Growth Factor (bFGF) (LaVail et al., Proc. Natl. Acad. Sci. U.S.A. 89, 11249-11253 (1992)), Pigment-Epithelium Derived Growth Factor (LaVail et al., Proc. Natl. Acad. Sci. U.S.A. 89, 11249-11253 (1992)) and Glial Cell Derived Neurotrophic factor (GDNF) (Hauck et al., Mol. Cell. Biol. 26, 2746-2757 (2006); Dalkara et al., Mol. Ther. 19, 1602-1608 (2011); Touchard et al., Gene Ther. 19, 886-898 (2012)). Among these, GDNF is of a particular interest due to its known broad spectrum of targets, including dopaminergic neurons, motorneurons of spinal cord, as well as retinal ganglion cells and photoreceptors of the eye. GDNF was first described in 1993 as a survival factor for midbrain dopaminergic neurons (Lin et al., Science 260, 1130-1132 (1993)) and since then has been shown to promote development (Rothermel, A. & Layer, P. G., Invest. Ophthalmol. Vis. Sci. 44, 2221-2228 (2003)), synaptogenesis (Ledda et al., Nat. Neurosci. 10, 293-300 (2007)) and survival of a wide variety of neurons (Saavedra, et al., Prog. Neurobiol. 86, 186-215 (2008)), including ganglion cells (Kyhn et al., Exp. Eye Res. 89, 1012-1020 (2009)) and photoreceptors (Politi et al., Invest. Ophthalmol. Vis. Sci. 42, 3008-3015 (2001)). Also, its overexpression is not known to cause deleterious significant side effects, such as electroretinogram depression described for CNTF (McGill et al., Invest. Ophthalmol. Vis. Sci. 48, 5756-5766 (2007)).

The mechanisms of GDNF delivery into the retina vary: it can be administered as recombinant protein in solution or in slow-delivery systems, it can be constitutively overexpressed in host cells by adeno-associated viruses and plasmids, or overexpressed in transplanted cells, such as neural progenitors or Schwann cells. These approaches come with two major limitations: lack of control over the expression levels and the targeted delivery of GDNF to the specific location within the retina. The subretinal injection of AAV or cells seems to solve the latter issue, resulting in significant, although only local, rescue of photoreceptors. The indirect neuroprotective approach, when a growth factor is induced in host cells, may potentially abrogate these problems. Indeed, GDNF induction in vitro has been extensively studied with multiple triggers identified: bFGF, dopamine, amitriptyline, valproic acid, rasagiline, among others (Saavedra et al., Prog. Neurobiol. 86, 186-215 (2008)). However, GDNF induction was observed at micro- to millimolar concentrations of the stimulating compounds, which complicates the translation of these findings to clinical treatments. The use of Compound I is associated with numerous advantages to earlier approaches and provides a solution to many of the drawbacks associated with earlier methods.

The results described herein represent a surprising discovery that the dopamine and serotonin-receptor antagonist Compound I induces GDNF production in retinal cells at concentrations as low as 30 nM. More than 2-fold induction was observed in all three of retinal systems tested: ARPE19, hRPC and mouse eyecups. Interestingly, the latter assay shows both the highest sensitivity (30 nM) and levels of GDNF (50 pg/ml) produced, which may be related to the natural tissue-like structure of the assay target. The induction of GDNF mRNA in vitro was significant, but lower compared to GDNF protein levels—which were increased 1.8 fold. The same ratio was observed in in vivo experiments in both wild-type and retinal degenerative (rhodopsin knockout) mice. A sustained release formulation was developed via suspension of Compound I particles that could be deposited in the vitreous and offer prolonged bioavailability of the compound. Pharmacokinetic studies showed a single 2 ul injection provides a sufficient amount of the compound locally for at least 2 weeks, and modeling shows that drug may be present for up to 4 weeks, resulting in prolonged GDNF induction. This upregulation is enough to rescue photoreceptors in rhodopsin knockout mice to a level that was previously achieved with the transplantation GDNF-overexpressing Schwann cells (Keegan et al., Invest. Ophthalmol. Vis. Sci. 44, 3526-3532 (2003)). It is also comparable to the success of AAV-mediated GDNF overexpression in rat models, such as S334ter (Dalkara et al., Mol. Ther. 19, 1602-1608 (2011)) and RCS (Touchard et al., Gene Ther. 19, 886-898 (2012)). However, this goal was achieved by a single intravitreal injection, which led to pan-retinal rescue which is more favorable in clinical applications.

This study demonstrates GDNF induction in vitro and in vivo by Compound I. The study also has shown that a single intravitreal injection of sustained release Compound I formulation is sufficient to rescue photoreceptors in rhodopsin-knockout retinal degeneration mice. Thus, Compound I is a potent neuroprotective compound that can induce GDNF in normal and diseased retina; this induction results in photoreceptor rescue in a mouse model of retinal degeneration; Compound I suspension provides prolonged (at least 2 weeks) sustainable release of Compound I after single intravitreal injection; and Compound I treatment results in appearance of ectopic photoreceptors in inner nuclear layer.

### Other Embodiments

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. Other implementations may be within the scope of the following claims.

The published patent and scientific literature referred to herein provides knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

1. A method of increasing glial cell-derived neurotrophic factor (GDNF) protein levels in a retina of a subject or decreasing retinal neuron loss in a retina of said subject, the method comprising:
administering Compound I, or a pharmaceutically acceptable salt thereof, to a subject, wherein the Compound I or pharmaceutically acceptable salt thereof increases GDNF protein levels in the retina or decreases retinal neuron loss in the retina.

2. The method of claim 1, wherein Compound I is administered locally.

3. The method of claim 1, wherein Compound I is administered by ocular delivery.

4. The method of claim 3, wherein Compound I is administered by intravitreal injection.

5. The method of claim 1, wherein Compound I is administered as a sustained release formulation.

6. (canceled)

7. The method of claim 1, wherein
(a) Compound I is administered locally;
(b) Compound I is administered by ocular delivery;
(c) Compound I is administered by intravitreal injection; or
(d) Compound I is administered as a sustained release formulation.

8.-10. (canceled)

11. The method of claim 6, wherein the retinal neuron is a photoreceptor, a ganglion cell, a horizontal cell, an amacrine cell, or a bipolar cell.

12.-15. (canceled)

16. A method of preventing or treating a retinal disorder in a subject, the method comprising:
administering Compound I, or a pharmaceutically acceptable salt thereof, to a subject in need thereof or at risk of developing a retinal disorder.

17. The method of claim 16, wherein the retinal disorder comprises a retinal degenerative disorder, retinal detachment, or retinal trauma.

18. The method of claim 17, wherein the retinal degenerative disorder comprises retinitis pigmentosa, age-related macular degeneration (AMD), glaucoma, diabetic retinopathy, retinopathy of prematurity, Usher syndrome, Stargardt's disease, Leber Congenital Amaurosis, choroideremia, Bardet-Biedl syndrome, or Refsum disease.

19. (canceled)

20. The method of claim 18, wherein the AMD is dry AMD or wet AMD.

21.-26. (canceled)

27. The method of claim 16, wherein
(a) Compound I is administered locally;
(b) Compound I is administered by ocular delivery; or
(c) Compound I is administered as a sustained release formulation.

28. (canceled)

29. (canceled)

30. The method of claim 16, wherein a second therapy for the retinal disorder is administered in combination with Compound I.

31. (canceled)

32. The method of claim 16, wherein the retinal disorder comprises a retinal degenerative disorder, retinal detachment, or retinal trauma.

33. The method of claim 32, wherein the retinal degenerative disorder comprises retinitis pigmentosa, age-related macular degeneration (AMD), glaucoma, diabetic retinopathy, retinopathy of prematurity, Usher syndrome, Stargardt's disease, Leber Congenital Amaurosis, choroideremia, Bardet-Biedl syndrome, or Refsum disease.

34. (canceled)

35. The method of claim 3, wherein the AMD is dry AMD or wet AMD.

36.-41. (canceled)

42. The method of claim 16, wherein
(a) Compound I is administered locally;
(b) Compound I is administered by ocular delivery
(c) Compound I is administered by intravitreal injection; or
(d) Compound I is administered as a sustained release formulation.

43.-45. (canceled)

46. The method of claim 16, wherein a second therapy for the retinal disorder is administered in combination with Compound I.

47. (canceled)

48. The method of claim 16, wherein the retinal disorder comprises a retinal degenerative disorder, retinal detachment, or retinal trauma.

49. The method of claim 48, wherein the retinal degenerative disorder comprises retinitis pigmentosa, age-related macular degeneration (AMD), glaucoma, diabetic retinopathy, retinopathy of prematurity, Usher syndrome, Stargardt's disease, Leber Congenital Amaurosis, choroideremia, Bardet-Biedl syndrome, or Refsum disease.

50. (canceled)

51. A method of increasing the number of retinal neurons in a retina of a subject, the method comprising:
administering Compound I, or a pharmaceutically acceptable salt thereof to a subject, wherein the Compound I or pharmaceutically acceptable salt thereof increases the number of retinal neurons in the retina, wherein the number of photoreceptors in the retina increases by at least about 0.5, 0.75, 1, 1.25, 1.5, 1.75, 2, 2.1, or 0.5-2.1 fold compared to the increase in the number of photoreceptors in the retina of a corresponding subject not administered the Compound I or pharmaceutically acceptable salt thereof.

52. The method of claim 51, wherein
(a) Compound I is administered locally;
(b) Compound I is administered by ocular delivery; or
(c) Compound I is administered as a sustained release formulation.

53. (canceled)

54. (canceled)

55. The method of claim 51, wherein the retinal neuron is a photoreceptor, a ganglion cell, a horizontal cell, an amacrine cell, or a bipolar cell.

56.-60. (canceled)

61. A composition comprising Compound I or a pharmaceutically acceptable salt thereof in an ophthalmically acceptable vehicle.

62. An eye drop composition, comprising the composition of claim 61 within a dispenser suitable for administering a drop of said composition to an eye of a subject.

63. A sustained release formulation comprising the composition of claim 61.

64. The composition of claim 63, comprising
(a) a polymer;
(b) a polymer, wherein the polymer comprises polyethylene glycol (PEG), poly(ethylene vinyl) acetate (EVA), superhydrolyzed PVA, hydroxyalkyl cellulose (HPC), methylcellulose (MC), hydroxypropyl methyl cellulose (HPMC), polycaprolactone, poly(glycolic) acid, poly(lactic) acid, or a polyanhydride;
(c) polyethylene glycol (PEG) having a molecular weight of at least about 2000, 2500, 3000, 3500, 4000, 5000, 6000, 7000, 8000, or 9000 Daltons (Da), or between about 2000 and about 10000 Da;
(d) mannitol;
(e) Compound I at a concentration of about 20, 25, 30, 35, 40, 45, 50, 55, 60, 65, 70, 75, or 80 mg/ml;
(f) about 10, 15, 20, 25, 30, 35, 40, or 10-40 mg/ml PEG;
(g) water;
(h) particles; or
(i) particles, wherein the size of the particles is: (i) D₁₀=about 0.5-10.0 μm; D₅₀=about 5-20 μm; D₉₀=about 10-40 μm; or (ii) D₁₀=about 6.8 μm; D₅₀=about 14.2 μm; D₉₀=about 26.5 μm.

65.-67. (canceled)

68. The composition of claim 63, which
(a) is a suspension;
(b) is formulated for injection into a vitreous chamber of a mammal;
(c) is formulated for injection at a dose of about 250-5000 μg or about 500-1000 μg of the composition into the vitreous chamber of the mammal; or
(d) is formulated such that an effective amount of Compound I is present in the vitreous chamber at least about 1, 2, 3, 4, 5, 6, 7, 8, 9, 10, 11, 12, 13, 14, or 1-14 days after the composition is injected into the vitreous chamber.

69.-75. (canceled)

76. The composition of claim 68, wherein the mammal is a human.

77. (canceled)


SEQUENCE LISTING (6 sequences)

SEQ ID NO: 1 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 2 - DNA, 3810 bp, Homo sapiens
ccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 3 - DNA, 4477 bp, Mus musculus
ggcaaacactctgttctcctctctcggctgttccgcgcttcttcttctcggtgctgggaaaacttctcggtgcaggctgcttgtcttctcgcccctgccttctgtcctgcagaaaaccgggagggcaggacactcggaggaggaaggacagccttcgaaggggacgaggaaagcgccgaggtagcctccagcaggcagagcgccctccctcggagcgccagacgcttttaccaagttgcgattgccccctggggctgctgtgagccgagctcagcgcacgttcctgtggatctaccctgctagaaaacgcgagccccgccgcggttcctgcaagccccgtgaagaggctctcggagtcacccactcgcaccccgcaccgggctcccgcagctccggtgccttttgcccggatctccgtccctcccccacactcaggccgccgttgccgccggaccccctcccctcccccctcccggttcggcgcccccctactccccaatcccggccctctggcccgggctctccacggccagcctgcgctcctggcgccctcatgtcttcaagggactgccagcgcggtttgacgtggtgtctcgttcggatctccaggcaagacctcggcagcatcagacaggcccgcctcgcactcgcagccaccgcgccccgaggaggtgcagagtgaggcgaccgcctcggagccacccgcagcgcccggggccccgtgctgctccagcccagagcgagcgccgcgcgcgcagccgtgccactcctcagccaaggcaggggcggctgctcagacttagtcttcttggggtccaggaggccggtgtgcgaggtgtcggcagccttcgccctcctcgcggcggcggcggcggcggctggcacggtggccgccgcctcctgcgtgtcctcgctgcccggcgcgacccctggattgcgtgctcttgctcccgaccttctgggcggggccccgcgctcctgcccgaggtccggatgggtctcctggatgggattcgggccacttggagttaatgtccaactgggggtctacggagaccggatccgaggtgccgccgccggacgggactctaagatgaagttatgggatgtcgtggctgtctgcctggtgttgctccacaccgcgtctgccttcccgctgcccgccggtaagaggcttctcgaagcgcccgctgaagaccactccctcggccaccgccgcgtgcccttcgcgctgaccagtgactccaatatgcctgaagattatcctgaccagtttgatgacgtcatggattttattcaagccaccattaaaagactgaaaaggtcaccagataaacaagcggcagcgcttcctcgaagagagaggaatcggcaggctgcagctgccagcccagagaattccagagggaaaggtcgcagaggccagaggggcaaaaatcgggggtgcgttttaactgccatacacttaaatgtcactgacttgggtttgggctatgaaaccaaggaggaactgatctttcgatattgcagcggttcctgtgaatcggccgagacaatgtatgacaaaatactaaaaaacctgtctcggagtagaaggctaacaagtgacaaagtaggccaggcatgttgcaggccggtcgccttcgacgacgacctgtcgtttttagatgacaacctggtttaccatattctaagaaagcattccgctaaacggtgtggatgtatctgaccccggctccagagactgctgtgtattgcattcctgctacagtgcgaagaaagggaccaaggttcccaggaaatgtttgcccagagtggaagataaggaccaagatggcggaggcagaggcagaagaagagaaaaagaaggaggaaggtagccaccgtgggagcctggtggaagaaggcccagctacagaaaactggataggagtgagaacagccgtctgagcacttcagggtggcagctgatgtcaccagaagctcagggctggtgtccttacttagctgttgccagtgtttatctgatacagtccatggtcaccgatggcggtccgagtcgtggatgcatgcaaagaaccaagccagtgtatctcctgtggtttgttttcacgtgtttgaagacgccagggaaatgtgacgattccggtgtcacctcttgtcactacattttactgctgaaaataagaaagatgtattttcctgtcactcaatggattcataccctggaaaggaaagggggtggggaggacaaaggcagagacgataaaaagatgacaacctttgaatttgaaaaccggggcctgaggtctattacatccagcattttgggggacgcttggtggttgattctggacacagagtgtgggggtatggagaagttggctaggaacccaaaaaaggctgtcctcatacttagaaacaaacctggaggcatctccttcgtgccctcagaaacaggaagcaagttgtggttttcggcagcattctcataggagagccaatggggaaagcccccagctgccccggccagggagaccccggggcctgttggtttacaaagacggatgttacatacacagctccgttcatgcgtggtcaccagtgaccacagaagctcctcgatgcaatgcatctgtttcaaatacagagatatagagaagatatttattgagattttaagttattattatttattaccattcactaatgaatatcttttttcttcccttatttattaaagtctcttttcaaaggtgccaaagtatatgtgctcacaaaatacaaaggtgacagaggaaatattaacattgagaacaagggtccattctcttcagagtataagagggtttttgctaggcaaaaaatcacttactttataaggcagtccttcactaactgccacagatttcagcatttccctccccctctttctgtggacaggatctcctgtagcctaggctgaccttgaacttactgcttggtcaaggacgaccttggccttctgatcctctgacttcctgtggtcttggcatggcagacatcaatcacaccgtatccacaggactaagtatagaattcagggcttcatccatgccaggcaactgtaggtaccaactgggttacacccagcccctgctttctatctgctaaaggaaagggtcaggagggccatctcagctccaccccctcactgggagtaccagtgggactgccacctgtttgcatctcctcttctatccatctgagcagcaaggccccagctccactgcccattgagttcaactctttttcccccttcatcatgggcaatatcataaccacgcattcagaaggctgaactgcccttggaagccctgaacatattgtcacctgaaagtgcaatgaaggcccctgacaggcagccagggtttaacagccccctcacccccccgggcaggtggtacctcttcctctatccttgcccacctgcttcgtccctatggttacacagacaggagaacataggggaactgtgcagggagaggccatctgccttgtcctttgccagggctcagttgtcacctgggctcaacttttgctaccccctcaaacccaatggattccaaggagaaagatggacaggaagagacatctgactggccatagaacaaagaagaggccctccctcattgtttggagggaagaagcagagggtgggaatggcaactctccctgcaccccataatgtccttgtctggcagccaacaaacaggtcatgccttgagtcctatgttagagccttgagtcctatgttacagatttcataaatggaatttttatgtagaagttaatgtatcactccctttgtggctgctatcccccgcccccagagtaggacagatagatctaaaataataaatgaccaattaattggcctctctcgaatagtcatgtcaaattttcaaagtaaccaagaggtaaaagttactaggtatcctttccccttccgtggccctaaagacccacgtttcgcatggttccacaccctctgcaggactgggaagtggagctacgtatgcgctgctctctccgctcctcctgctcaaaattgtgacaacctcagctgcccagcacatttcagatgtcgttccagaccctctgttcactccagagaatcctgaaagatgtggtagctgatgctcccaggtccagacacagcctctagctcttgggggaatccctggagaactcagcatctcgagcaggttcgaatgggagcaggctgccccacccagtccgcagcagggtaaagtttgcgacggttctttactgttgtgatcagcatctgccccagtaaagagcacacttctttggaagttctgacctctctgatgtctcggtcgtttgtgttatacaaccaaagttctctacaaactttatttttgtacaatatcattttgtaactttttacaaataaaagctcatttctattgct

SEQ ID NO: 4 - protein, 240 aa, Mus musculus
MGFGPLGVNVQLGVYGDRIRGAAAGRDSKMKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 5 - DNA, 700 bp, Rattus norvegicus
ggtctacggagaccggatccgaggtgccgccgccggacgggactctaagatgaagttatgggatgtcgtggctgtctgcctggtgttgctccacaccgcgtctgccttcccgctgcccgccggtaagaggcttctcgaagcgcccgccgaagaccactccctcggccaccgccgcgtgcccttcgcgctgaccagtgactccaatatgcccgaagattatcctgaccagtttgatgacgtcatggattttattcaagccaccatcaaaagactgaaaaggtcaccagataaacaagcggcggcacttcctcgaagagagaggaaccggcaagctgcagctgccagcccagagaattccagagggaaaggtcgcagaggccagaggggcaaaaatcgggggtgcgtcttaactgcaatacacttaaatgtcactgacttgggtttgggctacgaaaccaaggaggaactgatctttcgatattgtagcggttcctgtgaagcggccgagacaatgtacgacaaaatactaaaaaatctgtctcgaagtagaaggctaacaagtgacaaggtaggccaggcatgttgcaggccggtcgccttcgacgacgacctgtcgtttttagacgacagcctggtttaccatatcctaagaaagcattccgctaaacggtgtggatgtatctgaccctggctccagaga

SEQ ID NO: 6 - protein, 211 aa, Rattus norvegicus
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI